(19) United States
(12) Patent Application Publication
(10) Pub. No.: US 2023/0072852 A1
(43) Pub. Date: Mar. 9, 2023

(54) A SUTURING DEVICE

(71) Applicant: PALLIARE LIMITED, Galway (IE)

(72) Inventors: John O'DEA, Galway (IE); Hilary Elizabeth BARRETT, Limerick (IE); Robin Harold KENNEDY, Harrow (GB); Martin James Hamilton BRUGGEMANN, Laois (IE); Paul Maurice CREMIN, Dublin 14 (IE)

(73) Assignee: PALLIARE LIMITED, Galway (IE)

(21) Appl. No.: 17/795,297

(22) PCT Filed: Feb. 19, 2021
(86) PCT No.: PCT/IE2021/000001

§ 371 Date: Jul. 26, 2022

(30) Foreign Application Priority Data
Feb. 20, 2020 (IE) S2020/0031
Nov. 20, 2020 (IE) S2020/0262

(51) Int. Cl. A61B 17/12; A61B 17/04

(52) U.S. Cl. CPC A61B 17/12013; A61B 17/0483; A61B 17/0401; A61B 2017/00367

(57) ABSTRACT

A suturing device (1) for suturing parts (5,6) together of a lumen (8) with one or more sutures (3) comprises an actuator (34) having a handle (33) and first and second operating elements (60,68) slideable longitudinally on the handle (33). A tubular shield (35) extends from the handle (33), and a cannula (40) slideable in the tubular shield (35) is connected to the first operating element (60) for urging the cannula (40) between a withdrawn state within the tubular shield (35) to an extended state extending from the tubular shield (35). An elongated push rod (52) slideable in the cannula (40) terminating in a distal hook (55) is coupled to and operated by the second operating element (68). Anchor elements (22) of the suture (3) are located in the cannula (40), and are sequentially urged therefrom by the push rod (52), as the cannula (40) sequentially pierces through the parts (5,6). A loop (26) of the suture (3) is engaged by the hook (55), which is then withdrawn into the tubular shield (35) for engaging the adjustment element (25) of the suture (3) against an abutment face (38) of the tubular shield (35) for in turn drawing the anchor elements (22), and in turn the parts (5,6) of the colon (8) together. The adjustment element retains the parts (5,6) together.

The present invention relates to a suturing device for suturing two parts of a human or animal body together, and in particular, though not limited to a suturing device for suturing two parts of a lumen, vessel or organ together within a human or animal body. The invention also relates to a suture, and the invention relates to a cannula comprising the suture therein. The invention also relates to an actuator for operating the cannula for suturing, and to a combination of the cannula and the actuator whereby the cannula is releasably coupleable to the actuator. The invention also relates to a method for suturing two parts of a human or animal body together.

Complex polyps, which can be large and/or difficult to access, may be unsuitable for standard endoscopic excision techniques and thus require invasive surgical resection of the colon, ‘colectomy’. Colectomy normally removes approximately 30 cm of bowel and even when performed laparoscopically it is accompanied by an approximately 10% chance of serious and a 30% chance of less serious, postoperative complications. It has been suggested that a lesser procedure could be employed for the removal of such polyps, improving recovery due to less complications. To address this need, a hybrid, laparoendoscopic procedure which removes only a 6 cm disc of colon and has been developed. The procedure requires that the polyp or tumour be everted from within the colon to the outside wall of the colon (an endoscopic procedure), whereby it can then be removed by a surgical stapler (a laparoscopic procedure). This allows the user to treat patients presenting with complex polyps: defined as being large, or in adverse locations such as near the appendix orifice, ileocecal valve, or within a segment of diverticulosis. Currently no suitable apparatus is available for carrying out this procedure.

Accordingly, there is a need for an apparatus to address this problem.

The present invention is directed towards providing a suturing device for suturing two parts of a human or animal body together. The invention is also directed towards providing a suture, and the invention is further directed towards providing a cannula comprising a suture therein, and the invention is also directed towards providing an actuator for operating the cannula for suturing. The invention is also directed towards providing a combination of the cannula and the actuator whereby the cannula is releasably coupleable to the actuator. Further, the invention is directed towards providing a method for suturing two parts of a human or animal body together.

According to the invention there is provided a suturing device for applying a suture, the suture terminating at its opposite ends in respective anchor elements, the suturing device comprising:
a cannula having a bore extending longitudinally therethrough for accommodating the suture therein and terminating adjacent a distal end thereof in a piercing point,
an urging element urgeable into the cannula bore for urging the anchor elements of the suture sequentially from the cannula bore through the distal end thereof,
a first operating element coupled to the cannula, and
a second operating element coupled to the urging element,
the second operating element being operable for urging the urging element in the cannula bore in a distal direction to sequentially urge the anchor elements of the suture from the cannula.

In one embodiment of the invention an engagement element is provided for engaging a portion of the suture, and preferably, one of the first and second operating elements is operably coupled to the engagement element, and advantageously, an abutment element is provided for abutting an adjustment element of the suture for tightening thereof, and preferably, one of the first and second operating elements is operable for urging the engagement element in a proximal direction to withdraw a portion of the suture engaged by the engagement element through the adjustment element of the suture with the adjustment element abutting the abutment element to shorten the effective length of the suture between the anchor elements.

In another embodiment of the invention the cannula is non-releasably coupled to the first operating element, and in an alternative embodiment of the invention the cannula is releasably coupled to the first operating element.

In another embodiment of the invention the engagement element is non-releasably coupled to the one of the first and second operating elements, and in an alternative embodiment of the invention the engagement element is releasably coupled to the second operating element.

In another embodiment of the invention the urging element is urgeable in a distal direction relative to the cannula for urging the anchor elements of the suture from the cannula.

Preferably, the urging element is urgeable from a withdrawn state within the cannula bore in the distal direction relative to the cannula for urging the anchor elements of the suture from the cannula.

Preferably, in the withdrawn state of the urging element, a distal end thereof defines with the cannula a suture accommodating chamber for accommodating the respective anchor elements of the suture. Preferably, the suture accommodating chamber is defined in the cannula bore, and advantageously, the suture accommodating chamber is defined in the cannula bore between the distal end of the cannula and the distal end of the urging element.

Advantageously, the suture accommodating chamber is configured to accommodate the anchor elements of the suture sequentially along the suture accommodating chamber.

In one embodiment of the invention the length of the suture accommodating chamber is substantially equal to the sum of the lengths of the respective anchor elements of the suture.

In one embodiment of the invention a suture accommodating slot extends longitudinally into the cannula from the distal end thereof for accommodating a portion of the suture therethrough from the suture accommodating chamber.

In one embodiment of the invention the suture accommodating slot extends longitudinally in the cannula from the distal end thereof substantially the length of the suture accommodating chamber.

In one embodiment of the invention the urging element is urgeable from the withdrawn state distally through a first predefined distance for urging a first one of the anchor elements of the suture from the cannula.

In another embodiment of the invention the urging element is urgeable distally through a second predefined distance for urging a second one of the anchor elements of the suture from the cannula.

In one embodiment of the invention the urging element is urgeable sequentially through the first and second predefined distances.

In another embodiment of the invention the length of the first predefined distance is substantially equal to the length of the first one of the anchor elements of the suture to be urged from the cannula, and preferably, the length of the second predefined distance is substantially equal to the length of the second one of the anchor elements of the suture to be urged from the cannula.

In one embodiment of the invention the first and second predefined distances are substantially equal to each other.

In one embodiment of the invention the second operating element is operable from a first state thereof to a second state thereof for urging the urging element through the first predefined distance, and advantageously, the second operating element is moveable from the first state thereof to the second state thereof, and preferably, a first stop element is provided for terminating movement of the second operating element from the first state thereof to the second state thereof when the second operating element has reached the second state.

In another embodiment of the invention the second operating element is operable from the second state thereof to a third state thereof for urging the urging element through the second predefined distance, and preferably, the second operating element is moveable from the second state thereof to the third state thereof, and advantageously, a second stop element is provided for terminating movement of the second operating element from the second state thereof to the third state thereof when the second operating element has reached the third state thereof.

In another embodiment of the invention the second operating element is moveable from the first state thereof to the second state thereof through a third predefined distance for urging the urging element distally through the first predefined distance.

In another embodiment of the invention the second operating element is moveable from the first state thereof to the second state thereof with rectilinear motion. Alternatively, the second operating element is moveable from the first state thereof to the second state thereof with a rotary or a helical motion. Preferably, the second operating element is moveable from the first state thereof to the second state thereof relative to the first operating element.

Preferably, the second operating element is urgeable from the first state thereof to the second state thereof in a direction, which may be the same as or different to the direction through which the urging element is urged distally through the first predefined distance from the withdrawn state.

In one embodiment of the invention the second operating element is urgeable from the first state thereof to the second state thereof in a first direction.

In another embodiment of the invention the second operating element is operable from the second state thereof to the third state thereof through a fourth predefined distance for urging the urging element distally through the second predefined distance, and preferably, the second operating element is moveable from the second state thereof to the third state thereof.

In another embodiment of the invention the second operating element is moveable from the second state thereof to the third state thereof with rectilinear motion. In an alternative embodiment of the invention the second operating element is moveable from the second state thereof to the third state thereof with a rotary or a helical motion. Preferably, the second operating element is moveable from the second state thereof to the third state thereof relative to the first operating element.

In another embodiment of the invention the second operating element is urgeable from the second state thereof to the third state thereof in a direction, which may be the same as or different to the direction through which the urging element is urged distally through the second predefined distance.

In one embodiment of the invention the second operating element is moveable from the second state thereof to the third state thereof in the first direction.

Preferably, the second operating element is moveable from the second state thereof to the third state thereof in the first direction.

Preferably, a carrier is provided for carrying the first and second operating elements.

In one embodiment of the invention the carrier comprises a first carrier element and a second carrier element extending distally from the first carrier element.

In another embodiment of the invention the first carrier element is configured to carry the first and second operating elements.

In another embodiment of the invention the first carrier element forms a handle.

Preferably, the second carrier element terminates adjacent a distal end thereof in the abutment element for abutting the adjustment element of the suture.

In an alternative embodiment of the invention the distal end of the cannula defines the abutment element for abutting the adjustment element of the suture.

In one embodiment of the invention the second carrier element is configured to slideably accommodate the cannula therein, and preferably, the cannula is urgeable from a withdrawn state within the second carrier element to an extended state extending distally outwardly from the distal end of the second carrier element.

In one embodiment of the invention the second carrier element defines with the distal end of the cannula in the withdrawn state an adjustment element accommodating chamber for accommodating the adjustment element of the suture. Preferably, the adjustment element accommodating chamber is defined adjacent the distal end of the second carrier element.

In one embodiment of the invention the cannula is configured to urge the adjustment element of the suture from the adjustment element accommodating chamber through the distal end of the second carrier element as the cannula is urged from the withdrawn state to the extended state.

In another embodiment of the invention the first operating element is urgeable from a first state thereof to a second state thereof for urging the cannula from the withdrawn state within the second carrier element to the extended state.

In another embodiment of the invention the second operating element is urgeable with the first operating element when the first operating element is being urged from the first state thereof to the second state thereof for urging the cannula from the withdrawn state within the second carrier element to the extended state.

In another embodiment of the invention the first operating element is urgeable from the second state thereof to the first state thereof for urging the cannula from the extended state to the withdrawn state within the second carrier element. Preferably, the second operating element is urgeable with the first operating element when the first operating element is being urged from the second state thereof to the first state thereof for urging the cannula from the extended state to the withdrawn state within the second carrier element, and advantageously, the second operating element is urgeable from the third state thereof through the second state thereof to the first state thereof relative to the first operating element for urging the urging element in a proximal direction to the withdrawn state thereof.

Preferably, the second operating element is urgeable from the second state thereof to the first state thereof in a second direction. In one embodiment of the invention the second direction is different to the first direction, and preferably, the second direction is a direction which is opposite to the first direction.

In another embodiment of the invention the second operating element is urgeable from the third state thereof to the second state thereof in a direction which may be the same as or different to the second direction, and preferably, is a direction the same as the second direction.

In one embodiment of the invention the first operating element is urgeable from the second state thereof to the first state thereof in a direction which may be the same as or different to the second direction, and preferably, is the same as the second direction.

In one embodiment of the invention the second carrier element comprises an elongated tubular shield.

In one embodiment of the invention the elongated tubular shield comprises a flexible tubular shield, which preferably, is bendable, and advantageously, is sufficiently flexible and bendable to allow the tubular shield to be urged through a tortuous passageway, for example, through an artery, vein, colon and the like of a human or animal subject. In an alternative embodiment of the invention the tubular shield may comprise a rigid material.

In another embodiment of the invention the engagement element is coupled to the urging element adjacent the distal end thereof, and preferably, the urging element is urgeable through the cannula to an extended state with the engagement element located distally from the distal end of the cannula for engaging the portion of the suture.

In one embodiment of the invention the urging element is urgeable distally in the cannula to the extended state by the second operating element.

Preferably, the urging element is urgeable distally through a fifth predefined distance to the extended state after the urging element has been urged through the second predefined distance. Advantageously, the urging element is urgeable sequentially through the first, second and fifth predefined distances.

In another embodiment of the invention the second operating element is moveable from the third state thereof to a fourth state thereof for urging the urging element through the fifth predefined distance. Preferably, a third stop element is provided for terminating movement of the second operating element from the third state thereof to the fourth state thereof when the second operating element has reached the fourth state thereof.

In another embodiment of the invention the second operating element is moveable from the third state thereof to the fourth state thereof through a sixth predefined distance for urging the urging element distally through the fifth predefined distance.

In another embodiment of the invention the second operating element is urgeable from the third state thereof to the fourth state thereof relative to the first operating element. Preferably, the second operating element is urgeable from the third state thereof to the fourth state thereof with rectilinear motion. Alternatively, the second operating element is moveable from the third state thereof to the fourth state thereof with a rotary or a helical motion.

In one embodiment of the invention the second operating element is urgeable from the third state thereof to the fourth state thereof in a direction, which may be the same as or different to the first direction, and preferably, is a direction similar to the first direction.

The first predefined distance travelled by the urging element and the third predefined distance travelled by the second operating element may be the same or different, and in general when the second operating element is being urged between the first and second states thereof with rectilinear motion, the third predefined distance travelled by the second operating element will be similar to the first predefined distance travelled by the urging element.

The second predefined distance travelled by the urging element and the fourth predefined distance travelled by the second operating element may be the same or different, and in general when the second operating element is being urged between the second and third states thereof with rectilinear motion, the fourth predefined distance travelled by the second operating element will be similar to the second predefined distance travelled by the urging element.

The fifth predefined distance travelled by the urging element and the sixth predefined distance travelled by the second operating element may be the same or different, and in general when the second operating element is being urged between the third and fourth states thereof with rectilinear motion, the sixth predefined distance travelled by the second operating element will be similar to the fifth predefined distance travelled by the urging element.

In one embodiment of the invention the second operating element is operable for urging the urging element in a proximal direction from the extended state for urging the engagement element in a proximal direction, for in turn urging the portion of the suture through the adjustment element.

In one embodiment of the invention the second operating element is operable in the second direction for urging the urging element in the proximal direction from the extended state for urging the engagement element in the proximal direction.

In another embodiment of the invention the first operating element is urgeable with the second operating element in the same direction in which the second operating element is travelling as the second operating element is being urged for urging the urging element from the extended state, and preferably, the first operating element is urgeable in the second direction with the second operating element as the second operating element is being urged in the second direction for urging the urging element from the extended state.

In one embodiment of the invention the second operating element is moveable relative to the first operating element for urging the urging element in a proximal direction to the withdrawn state.

In another embodiment of the invention the urging element is urgeable from the extended state for in turn urging the engagement element proximally for urging the adjustment element of the suture into abutting engagement with the abutment element as the portion of the suture engaged by the engagement element is urged proximally through the adjustment element by the engagement element.

In one embodiment of the invention the engagement element is connected to the second operating element, and in another embodiment of the invention the engagement element is releasably connected to the second operating element. In an alternative embodiment of the invention the engagement element is non-releasably connected to the second operating element.

In a further alternative embodiment of the invention the engagement element is coupled to the first operating element, and the first operating element is urgeable from the second state thereof to the first state thereof for urging the engagement element in a proximal direction for drawing the portion of the suture through the adjustment element.

Preferably, the engagement element is releasably coupleable to the first operating element.

In another embodiment of the invention the engagement element comprises a hook for engaging a loop of the suture for urging the loop of the suture through the adjustment element.

In one embodiment of the invention the first operating element is slideably mounted on the first carrier element, and preferably, is slideable longitudinally on the first carrier element in the first and second directions.

In another embodiment of the invention the first operating element comprises a first body member having a first bore extending therein, which preferably, is slideably engageable with the first carrier element, and preferably, is slideable in the first and second directions relative to the first carrier element.

In another embodiment of the invention the second operating element is slideably mounted on the first operating element, and preferably, is slideable longitudinally on the first operating element in the first and second directions. Advantageously, the second operating element is rotatable on the first operating element. Preferably, the second operating element comprises a second body member having a second bore extending therein, and advantageously, the second bore is slideably engageable with the first operating element, and preferably, is slideable in the first and second directions relative to the first operating element.

In another embodiment of the invention a guide means is provided for guiding movement of the second operating element relative to the first operating element between the first and second states of the second operating element, between the second and third states of the second operating element, and between the third and fourth states of the second operating element, and preferably, for guiding movement of the second operating element relative to the first operating element through the third and fourth predefined distances, and advantageously, through the sixth predefined distance.

In one embodiment of the invention the guide means is configured to guide the second operating element to move relative to the first operating element in a third direction different to the first and second direction between the travel of the second operating element relative to the first operating element through the third predefined distance and the fourth predefined distance.

Advantageously, the guide means is configured to guide the second operating element relative to the first operating element in the third direction or in a fourth direction different to the first and second direction between the travel of the second operating element relative to the first operating element through the fourth predefined distance and the sixth predefined distance.

In one embodiment of the invention the third direction extends transversely of the first direction, and preferably, the third direction extends transversely of the longitudinal direction of movement of the second operating element relative to the first operating element in the first direction.

In another embodiment of the invention the fourth direction extends transversely of the first direction.

Preferably, the third direction of movement of the second operating element relative to the first operating element is in a circumferential direction relative to the first operating element, and advantageously, the fourth direction of movement of the second operating element relative to the first operating element is in a circumferential direction relative to the first operating element.

In one embodiment of the invention the fourth direction is in the same direction as the third direction. Alternatively, the fourth direction is a direction opposite to the third direction.

In another embodiment of the invention the guide means comprises a camming groove located on one of the first and second operating elements, and a cam follower located on the other one of the first and second operating elements. In one embodiment of the invention the camming groove comprises first and second longitudinally extending camming segments corresponding to the travel of the second operating element relative to the first operating element in the first direction through the third and fourth predefined distances, and a first intermediate camming segment joining the first and second camming segments. Preferably, the first intermediate camming segment extends in a circumferential direction relative to the first and second camming segments. Advantageously, the second longitudinally extending camming segment is offset circumferentially from the first longitudinally extending camming segment.

In a further embodiment of the invention the camming groove comprises a third longitudinally extending camming segment corresponding to the travel of the second operating element relative to the first operating element in the first direction through the sixth predefined distance, and advantageously, a second intermediate camming segment joins the second and third longitudinally extending camming segments. In one embodiment of the invention a second intermediate camming segment extends in a circumferential direction relative to the second and third longitudinally extending camming segments, and preferably, the third longitudinally extending camming segment is offset circumferentially from the second camming segment.

In one embodiment of the invention the camming groove is formed in the first operating element, and advantageously, the cam follower extends from the second operating element into engagement with the camming groove.

In another embodiment of the invention the first operating element is keyed relative to the first carrier element for preventing rotation of the first operating element relative to the first carrier element.

In another embodiment of the invention the first operating element is coaxial with the first carrier element, and preferably, the second operating element is coaxial with the first operating element.

In another embodiment of the invention the urging element comprises an elongated push rod extending from the second operating element.

In one embodiment of the invention the push rod comprises a flexible material, and preferably, a flexible bendable material, and advantageously, the flexible bendable material of the push rod is sufficiently flexible and bendable to allow the push rod to bend and flex as the second carrier element is being urged through a tortuous passageway, for example, through an artery, a vein, a colon or the like of a human or animal subject. In an alternative embodiment of the invention the push rod may comprise a rigid material.

In another embodiment of the invention the cannula is coupled to the first operating element by an elongated tubular connecting member, and preferably, the urging element extends through the tubular connecting member.

In one embodiment of the invention the cannula comprises an elongated flexible member, and advantageously, an elongated bendable flexible member, and advantageously, the cannula is sufficiently flexible and bendable for urging through a tortuous passageway, for example, an artery, vein or the colon of a human or animal subject. Preferably, the distal most part of the cannula is rigid for facilitating puncturing of tissue or other material of a human or animal subject.

In a further alternative embodiment of the invention the cannula may comprise a short cannula of a rigid material, and preferably, the short cannula may be connected to the first operating element through an elongated flexible tubular member, the elongated flexible tubular member being bendable, and being sufficiently flexible and bendable for urging through a tortuous passageway, for example, through an artery, vein or the colon of a human or animal subject.

In an alternative embodiment of the invention the cannula may comprise a rigid material.

In another embodiment of the invention the cannula is releasably coupleable to the tubular connecting member.

In another embodiment of the invention the engagement element is coupled to the tubular connecting member.

In a further embodiment of the invention the engagement element is releasably coupleable to the tubular connecting member.

The invention also provides a suture terminating at respective opposite ends thereof in respective anchor elements and configured to form a loop extending from one of the anchor elements to the other one of the anchor elements, and an adjustment element engageable with respective legs of the loop for adjusting the effective length of the suture extending between the anchor elements.

Preferably, the adjustment element comprises a retainer configured for accommodating movement of at least one of the legs of the loop therethrough in a direction for urging the adjustment element along that leg of the loop towards the corresponding anchor element and for retaining the adjustment element engaged with that leg of the loop at a selected distance from the corresponding anchor element.

Advantageously, the retainer comprises a pair of spaced apart abutment members defining a suture accommodating gap therebetween for accommodating the at least one leg of the loop therethrough.

Preferably, the abutment members are configured to permit the movement of the at least one leg of the loop through the suture accommodating gap in the direction for urging the adjustment element towards the corresponding anchor element.

Preferably, the abutment members are configured for engaging the at least one leg of the loop in the suture accommodating gap with the adjustment element at the selectable distance from the corresponding anchor element.

Preferably, the abutment members are configured to engage the at least one leg of the loop in the suture accommodating gap, and preferably, the abutment members are configured to engage the at least one leg of the loop in the suture accommodating gap with an engagement force, and preferably, the engagement force acts on the at least one leg of the suture in a generally transverse direction, and advantageously, the engagement force is of a predefined engagement force value.

Preferably, the predefined engagement force value is configured to prevent movement of the at least one leg of the loop through the suture accommodating gap until a pulling force acting on the at least one leg of the loop of the suture to urge the at least one leg through the suture accommodating gap exceeds a predefined pulling force value.

In another embodiment of the invention at least one of the abutment members is configured to engage the at least one leg of the loop in the suture accommodating gap with a friction grip, and preferably, the at least one of the abutment members is configured to apply a friction force to the at least one leg of the loop in the suture accommodating gap.

In one embodiment of the invention at least one of the abutment members is resiliently biased towards the other one thereof, and advantageously, the abutment members are resiliently biased towards each other.

In another embodiment of the invention at least one of the abutment members is spring biased towards the other one of the abutment members, and preferably, the abutment members are spring biased towards each other.

In an alternative embodiment of the invention a gripping element is provided on at least one of the abutment members for gripping the at least one leg of the loop in the suture accommodating gap. Preferably, the gripping element is configured to prevent movement of the adjustment element along the at least one leg of the loop in the direction away from the corresponding anchor element, and advantageously, the gripping element is configured to permit movement of the adjustment element along the at least one leg of the loop in the direction towards the corresponding anchor element.

Preferably, the adjustment element comprises a first cross-member extending between and joining the abutment members. Advantageously, the first cross-member has a suture accommodating opening therethrough for accommodating the at least one leg of the loop into or out of the suture accommodating gap.

In one embodiment of the invention the first cross-member is located relative to the abutment members, so that the suture accommodating opening therein accommodates the at least one leg of the loop from the suture accommodating gap.

In an alternative embodiment of the invention the first cross-member is located relative to the abutment members so that the suture accommodating opening therein accommodates the at least one leg of the loop into the suture accommodating gap.

Advantageously, a second cross-member extends between and joins the abutment members. Preferably, the second cross-member has a suture accommodating opening therethrough for accommodating the at least one leg of the loop into the suture accommodating gap.

In one embodiment of the invention each cross-member is of arcuate shape, and preferably, is of concave arcuate shape when viewed from a direction through the suture accommodating gap.

Preferably, each cross-member is configured to resiliently or spring urge the abutment members towards each other.

Preferably, the suture accommodating opening of each cross-member is configured to accommodate the two legs of the loop of the suture therethrough.

In one embodiment of the invention at least one of the abutment members defines one of an abutment face and an abutment edge for engaging the at least one leg of the loop, and preferably, each of the abutment members defines a corresponding abutment face for engaging the at least one leg of the loop of the suture.

In an alternative embodiment of the invention, at least one of the abutment members defines an abutment edge for engaging the at least one leg of the loop of the suture, and in another embodiment of the invention each abutment member defines a corresponding abutment edge for engaging the at least one leg of the loop of the suture.

In a further alternative embodiment of the invention, one of the abutment members comprises an abutment face, and the other one of the abutment members comprises an abutment edge cooperating with the abutment face of the other abutment member for engaging the at least one leg of the loop therebetween.

In another embodiment of the invention the retainer is configured for accommodating the two legs of the loop of the suture therethrough.

Preferably, the suture accommodating gap defined between the pair of the abutment members is configured for accommodating the two legs of the loop of the suture therethrough.

In one embodiment of the invention the suture is severed intermediate the ends thereof, one of the legs of the suture adjacent the severed portion being non-adjustably secured to the adjustment element, and the other one of the legs of the suture extending through the retainer of the adjustment element.

In one embodiment of the invention the adjustment element defines a transverse cross-section of shape corresponding to half of a figure of eight or a half of an hourglass, the arcuate part of the half of the figure of eight or the half of the hourglass being defined by the first cross-member, and side parts of the half of the figure of eight or the hourglass being defined by the respective abutment members.

In another embodiment of the invention the adjustment element defines a transverse cross-section of a shape corresponding to a figure of eight or an hourglass, the upper and lower arcuate parts of the figure of eight or the hourglass being defined by the first and second cross-members, and the side parts of the figure of eight or the hourglass being defined by the abutment members.

Preferably, each anchor element comprises a transverse member secured to the corresponding end of the suture, and extending transversely of the suture.

Preferably, each end of the suture terminates in the corresponding transverse member intermediate the ends of the transverse member, and preferably, midway between the ends of the transverse member.

In another embodiment of the invention each transverse member comprises a transverse bar.

In one embodiment of the invention the suture comprises a dissolvable material configured for dissolving in a human or animal body. Advantageously, the anchor elements comprise a dissolvable material, and are configured for dissolving in a human or animal body.

Preferably, the adjustment element comprises a dissolvable material configured for dissolving in a human or animal body.

In one embodiment of the invention the suture comprises a medical grade material, and in another embodiment of the invention the suture comprises a nylon material, although the suture may be of any suitable material.

In one embodiment of the invention each anchor element comprises a medical grade material, and in another embodiment of the invention each anchor element comprises a stainless steel material, although each anchor element may be of any suitable material.

In a further embodiment of the invention the adjustment element comprises a medical grade material, and in another embodiment of the invention the adjustment element comprises a stainless steel material, although the adjustment element may be of any suitable material.

In a further embodiment of the invention the suture is configured for joining two parts of tissue of a human or animal subject together, and preferably, the predefined pulling force is greater than the force induced in the at least one leg of the loop between the corresponding anchor element and the adjustment element by the two parts of the tissue acting on the respective anchor elements when the two parts of the tissue are joined together by the suture.

Further the invention provides the adjustment element of the suture.

The invention also provides a cannula having a bore extending longitudinally therethrough, and terminating adjacent a distal end thereof in a piercing point, and a suture accommodating chamber for accommodating a suture according to the invention therein formed in the cannula bore.

Preferably, the suture accommodating chamber is located adjacent the distal end of the cannula.

Preferably, the suture is located in the suture accommodating chamber with the anchor elements arranged sequentially longitudinally in the suture accommodating chamber.

Preferably, the anchor elements of the suture are located in the suture accommodating chamber parallel thereto.

In one embodiment of the invention the length of the suture accommodating chamber is substantially equal to the sum of the lengths of the anchor elements of the suture.

In another embodiment of the invention a suture accommodating slot extends into the cannula from the distal end thereof, and preferably, the suture accommodating slot extends from the distal end of the cannula in a proximal direction, and advantageously, the suture accommodating slot extends into the cannula from the proximal end of the piercing point, and preferably, the suture accommodating slot extends the length of the suture accommodating chamber.

In one embodiment of the invention the transverse cross-section of the suture accommodating chamber is just greater than the transverse cross-section of the anchor elements.

In another embodiment of the invention the cannula is of circular external transverse cross-section, and preferably, the suture accommodating chamber is of circular transverse cross-section.

In another embodiment of the invention the transverse cross-section of the suture accommodating chamber is substantially similar to the transverse cross-section of the cannula bore.

In one embodiment of the invention the proximal end of the cannula is configured for receiving an urging element into the cannula bore for urging the anchor elements of the suture from the suture accommodating chamber through the distal end of the cannula.

In one embodiment of the invention the proximal end of the cannula is configured for coupling to a first operating element of an actuator, and advantageously, the proximal end of the cannula is configured for releasably coupling to the first operating element of the actuator.

In another embodiment of the invention the proximal end of the cannula is configured for coupling to a connecting element for connecting the cannula to the first operating element of the actuator, and preferably, for releasably coupling the cannula to the connecting element of the actuator.

In one embodiment of the invention the proximal end of the cannula is configured for coupling to an elongated tubular connecting member for connecting the cannula to the first operating element of the actuator.

Further the invention provides an actuator of a suturing device, the actuator comprising a first operating element configured for releasably coupling to the cannula according to the invention, a second operating element having an urging element, or being coupleable with an urging element for extending into the cannula bore from the proximal end thereof when the cannula is coupled to the first operating element, the second operating element being operable for urging the urging element in a distal direction into the suture accommodating chamber of the cannula for urging the anchor elements of the suture sequentially therefrom through the distal end of the cannula.

In one embodiment of the invention the second operating element is configured for urging the urging element distally in the suture accommodating chamber through a first predefined distance for urging a distal most one of the anchor elements of the suture from the suture accommodating chamber through the distal end of the cannula.

In another embodiment of the invention the second operating element is configured for urging the urging element distally through a second predefined distance in the suture accommodating chamber for urging a proximal most one of the anchor elements of the suture from the suture accommodating chamber through the distal end of the cannula.

In one embodiment of the invention the second operating element is operable from a first state thereof to a second state thereof for urging the urging element through the first predefined distance, and advantageously, is moveable from the first state thereof to the second state thereof, and preferably, is moveable from the first state thereof to the second state thereof with rectilinear motion. Alternatively, the second operating element is moveable from the first state thereof to the second state thereof with a rotary or a helical motion. Preferably, a first stop element is provided for terminating movement of the second operating element from the first state thereof to the second state thereof when the second operating element has reached the second state thereof.

In another embodiment of the invention the second operating element is operable from the second state thereof to a third state thereof for urging the urging element through the second predefined distance, and advantageously, the second operating element is moveable from the second state thereof to the third state thereof, and preferably, the second operating element is moveable from the second state thereof to the third state thereof with rectilinear motion. Alternatively, the second operating element is moveable from the second state thereof to the third state thereof with a rotary or a helical motion. Preferably, a second stop element is provided for terminating movement of the second operating element from the second state thereof to the third state thereof when the second operating element has reached the third state thereof.

Preferably, the second operating element is urgeable from the first state thereof to the second state thereof through a third predefined distance for urging the urging element distally through the first predefined distance.

In another embodiment of the invention the second operating element is urgeable from the second state thereof to the third state thereof through a fourth predefined distance for urging the urging element distally through the second predefined distance.

In another embodiment of the invention the second operating element is moveable relative to the first operating element between the first and second states of the second operating element.

Advantageously, the second operating element is moveable relative to the first operating element between the second and third states of the second operating element.

In one embodiment of the invention the second operating element is moveable in a first direction from the first state thereof to the second state thereof, and preferably, the second operating element is moveable from the second state thereof to the third state thereof in a direction which may be the same as or different to the first direction, and preferably, is the same as the first direction.

In another embodiment of the invention the actuator further comprises a carrier for carrying the first and second operating elements. Preferably, the carrier comprises a first carrier element, and advantageously, the carrier comprises a second carrier element extending distally from the first carrier element. Preferably, the first carrier element is configured for carrying the first and second operating elements. Advantageously, the second carrier element is configured for accommodating the cannula therein, and preferably, for slideably accommodating the cannula therein.

In another embodiment of the invention the first operating element is moveable from a first state thereof to a second state thereof for urging the cannula distally from a withdrawn state located within the second carrier element to an extended state with the cannula extending distally outwardly from a distal end of the second carrier element.

In another embodiment of the invention the first operating element is slideable relative to the first carrier element between the first and second states thereof.

In a further embodiment of the invention an engagement element is provided for engaging a portion of the suture and for urging the portion of the suture through an adjustment element of the suture. Preferably, the engagement element is connectable to one of the first and second operating elements, and in one embodiment of the invention is releasably connectable to the one of the first and second operating elements.

In another embodiment of the invention the engagement element is connectable to the second operating element through the urging element, and preferably, the engagement element is located adjacent the distal end of the urging element.

In one embodiment of the invention the engagement element is urgeable by the one of the first and second operating elements distally outwardly through the distal end of the second carrier element for engaging the portion of the suture.

In another embodiment of the invention the engagement element is urgeable in a proximal direction relative to the second carrier element by the one of the first and second operating elements for urging the portion of the suture through the adjustment element with the adjustment element abutting an abutment element of the actuator, which preferably, is defined by the distal end of the second carrier element.

In another embodiment of the invention the second operating element is operable from the third state thereof to a fourth state thereof for urging the urging element distally through the cannula to an extended state for urging the engagement element through the distal end of the cannula and outwardly therefrom.

Preferably, the urging element is urgeable through a fifth predefined distance to the extended state after the second predefined distance for urging the engagement element distally through the distal end of the cannula and outwardly therefrom.

Preferably, the urging element is urgeable sequentially through the first, second and fifth predefined distances.

Advantageously, the second operating element is moveable from the third state thereof to the fourth state thereof through a sixth predefined distance for urging the urging element distally through the fifth predefined distance.

Preferably, a third stop element is provided for terminating movement of the second operating element from the third state thereof to the fourth state thereof when the second operating element has reached the fourth state thereof.

In one embodiment of the invention the second operating element is moveable from the third state thereof to the fourth state thereof in a direction which may be the same as or different to the first direction, and preferably, is the same as the first direction.

In another embodiment of the invention the second operating element is moveable in a second direction from the second state thereof to the first state thereof, and preferably, is also moveable from the third state thereof to the second state thereof in a direction which may be the same as or different to the second direction, and preferably, is the same as the second direction. Advantageously, the second operating element is moveable from the fourth state thereof to the third state thereof in a direction which may be the same as or different to the second direction, and preferably, is the same as the second direction. In one embodiment of the invention the second direction is opposite to the first direction.

In another embodiment of the invention a guide means is provided for guiding movement of the second operating element relative to the first operating element from the first state thereof to the second state thereof, and preferably, from the second state thereof to the third state thereof, and advantageously, from the third state thereof to the fourth state thereof.

In one embodiment of the invention the guide means is adapted to guide the second operating element to move relative to the first operating element in a third direction different to the first direction between travel of the second operating element through the third predefined distance and the fourth predefined distance.

Advantageously, the guide means is adapted to guide the second operating element relative to the first operating element in the third direction or a fourth direction different to the first direction between travel of the second operating element through the fourth predefined distance and the sixth predefined distance.

In one embodiment of the invention the third direction is in a direction transversely of the first direction, and preferably, the third direction is in a direction transversely of the longitudinal direction of movement of the second carrier element relative to the first carrier element in the first direction.

In another embodiment of the invention the fourth direction is in a direction transversely of the first direction.

Preferably, the third direction of movement of the second operating element relative to the first operating element is in a circumferential direction relative to the first operating element, and advantageously, the fourth direction of movement of the second operating element relative to the first operating element is in a circumferential direction relative to the first operating element.

In one embodiment of the invention the fourth direction is in a direction of one of the same direction as the third direction of travel and opposite to the third direction of travel.

In another embodiment of the invention the guide means comprises a camming means, and preferably, the camming means comprises a camming groove located on one of the first and second operating elements, and a cam follower located on the other one of the first and second operating elements. In one embodiment of the invention the camming groove comprises first and second longitudinally extending camming segments corresponding to the travel of the second operating element relative to the first operating element in the first direction through the third and fourth predefined distances, and a first intermediate camming segment joining the first and second camming segments. Preferably, the first intermediate camming segment extends in a circumferential direction relative to the first and second camming segments. Advantageously, the second longitudinally extending camming segment is offset circumferentially from the first longitudinally extending camming segment.

In a further embodiment of the invention the camming groove comprises a third longitudinally extending camming segment corresponding to the travel of the second operating element relative to the first operating element in the first direction through the sixth predefined distance, and advantageously, a second intermediate camming segment joins the second and third longitudinally extending camming segments. In one embodiment of the invention the second intermediate camming segment extends in a circumferential direction relative to the second and third longitudinally extending camming segments, and preferably, the third longitudinally extending camming segment is offset circumferentially from the second longitudinally extending camming segment.

In one embodiment of the invention the camming groove is formed in the first operating element, and advantageously, the cam follower extends from the second operating element into engagement with the camming groove.

In one embodiment of the invention the first operating element comprises a first body member having a first bore extending therein, the first carrier element being longitudinally slideable in the first bore of the first body member for accommodating slideable movement of the first operating element in the first and second directions relative to the first carrier element.

In another embodiment of the invention the second operating element comprises a second body member having a second bore extending therein, the first body member being longitudinally slideable in the second bore of the second body member for accommodating slideable movement of the second operating element relative to the first operating element in the first and second directions. Preferably, the second body member is rotatable on the first body member to accommodate rotational movement of the second operating element.

In one embodiment of the invention the first operating element is keyed relative to the first carrier element for preventing rotation of the first operating element on the first carrier element.

In another embodiment of the invention the first operating element is coaxial with the first carrier element, and preferably, the second operating element is coaxial with the first operating element.

In another embodiment of the invention the urging element comprises an elongated push rod coupleable to the second operating element.

In another embodiment of the invention an elongated tubular connecting member is coupleable to the first operating element, and preferably, the tubular connecting member terminates in a coupling element for coupling the cannula to the tubular connecting member.

In another embodiment of the invention the urging element extends through the tubular connecting member.

In one embodiment of the invention the engagement element is coupleable to the tubular connecting member, and preferably, is releasably coupleable to the tubular connecting member.

In another embodiment of the invention the engagement element is coupleable to the urging element, and preferably, is releasably coupleable to the urging element.

In one embodiment of the invention the cannula is of circular transverse cross-section, and preferably, the cannula bore is of circular transverse cross-section.

In one embodiment of the invention the first carrier element is of external circular transverse cross-section, and preferably, the second carrier element is of circular external transverse cross-section, and advantageously, the cannula accommodating bore extending through the second carrier element is of circular transverse cross-section.

In another embodiment of the invention the first carrier element defines a longitudinally extending bore extending longitudinally therethrough for accommodating the urging element and the one of the cannula and the tubular connecting member to the first and second operating elements.

The invention also provides a suturing device comprising an actuator according to the invention and a cannula according to the invention having a suture according to the invention located therein, the cannula being releasably coupled to the actuator.

The invention further provides a method for suturing first and second parts of a lumen, vessel or organ in a cavity of a human or animal body using the suturing device according to the invention, the method comprising urging the cannula into the cavity or into the lumen, vessel or organ with a suture according to the invention located in the suture accommodating chamber of the cannula, urging the distal piercing tip of the cannula through the tissue of the first part in the lumen, vessel or organ, urging the urging element distally into the suture accommodating chamber for urging a distal most one of the anchor elements of the suture through the distal end of the cannula, withdrawing the cannula from the first part in the lumen, vessel or organ leaving the suture extending through the first part from the anchor element from the distal most one of the anchor elements, urging the distal piercing point of the cannula through the second part in the lumen, vessel or organ, urging the urging element distally in the suture accommodating chamber for urging the proximal most one of the anchor elements through the distal end of the cannula, withdrawing the cannula from the second part of the lumen, vessel or organ, leaving the suture extending through the second part from the second anchor element, engaging the engagement element with the suture and urging the suture through the adjustment element of the suture for reducing the effective length of the suture between the anchor elements to pull the first and second parts to be sutured together.

Preferably, the engagement element is disengaged from the suture after the first and second parts of the lumen, vessel or organ have been urged together, and the cannula is withdrawn from the cavity or the lumen, vessel or organ.

In one embodiment of the invention the adjustment element of the suture is urged against the abutment element as the suture is being urged through the adjustment element by the engagement element.

Preferably, the cannula is initially located within the second carrier element in the withdrawn state, and the second carrier element is urged into the cavity or the lumen, vessel or organ before the cannula is urged from the withdrawn state through the distal end of the second carrier element.

Preferably, the engagement element is urged distally outwardly from the cannula prior to engaging the portion of the suture to be urged through the adjustment element.

Advantageously, the engagement element is urged into the second carrier element for drawing the portion of the suture into the second carrier element with the adjustment element of the suture abutting the abutment element defined by the distal end of the second carrier element for urging the portion of the suture through the adjustment element.

In another embodiment of the invention the method according to the invention is configured for suturing first and second parts of a colon wall of a colon on respective opposite sides of a polyp together, for everting the polyp so that the polyp extends from an external side of the colon wall.

The invention also provides a suturing device for use in a method for suturing a first part of a lumen, vessel or organ in a cavity of a human or animal body to a second part of the lumen, vessel or organ.

The advantages of the invention are many. The suturing device according to the invention enables suturing to be carried out in or on a lumen, vessel or organ in a minimally invasive procedure. In particular, the suturing device according to the invention enables two parts of a lumen, vessel or organ to be sutured together, so that a part of the lumen, vessel or organ between the sutured parts may be everted outwardly of the vessel, so that the everted part of the lumen, vessel or organ can be subsequently removed externally of the lumen, vessel or organ. The suturing device according to the invention enables the suturing procedure to be carried out within the lumen vessel or organ, or externally of the lumen vessel or organ, in a cavity, in which the lumen, vessel or organ is located, such as the abdominal cavity. In a procedure where the suturing of the two parts of the lumen, vessel or organ is carried out externally of the lumen, vessel or organ, for example, in the abdominal cavity, the everted part of the lumen, vessel or organ is located internally in the lumen, vessel or organ, and can be readily removed from within the lumen, vessel or organ.

A particularly important advantage provided by the suturing device according to the invention is in its use in a procedure for everting a polyp or a complex polyp in the colon. Typically, it is desirable that the polyp be everted externally of the colon wall, so that it can subsequently be removed by a laparoscopic procedure carried out in the abdominal cavity. The suturing device according to the invention enables the parts of the colon wall in which the polyp is located to be sutured together with the suturing operation being carried out within the colon, typically, with the suturing device having been rectally entered into the colon. Once the polyp has been everted externally onto the external side of the colon wall, the polyp can then be readily removed by stapling which can be carried out by a relatively straightforward laparoscopic procedure carried out in the abdominal cavity.

The invention will be more clearly understood from the following description of some preferred embodiments thereof which are given by way of example only with reference to the accompanying drawings, in which:

FIG. 1 is a perspective view of a portion of a suturing device according to the invention,

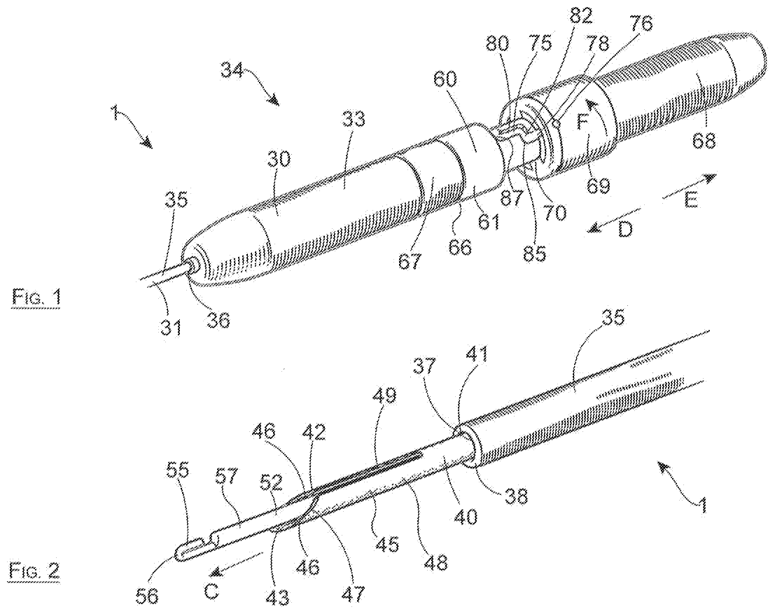

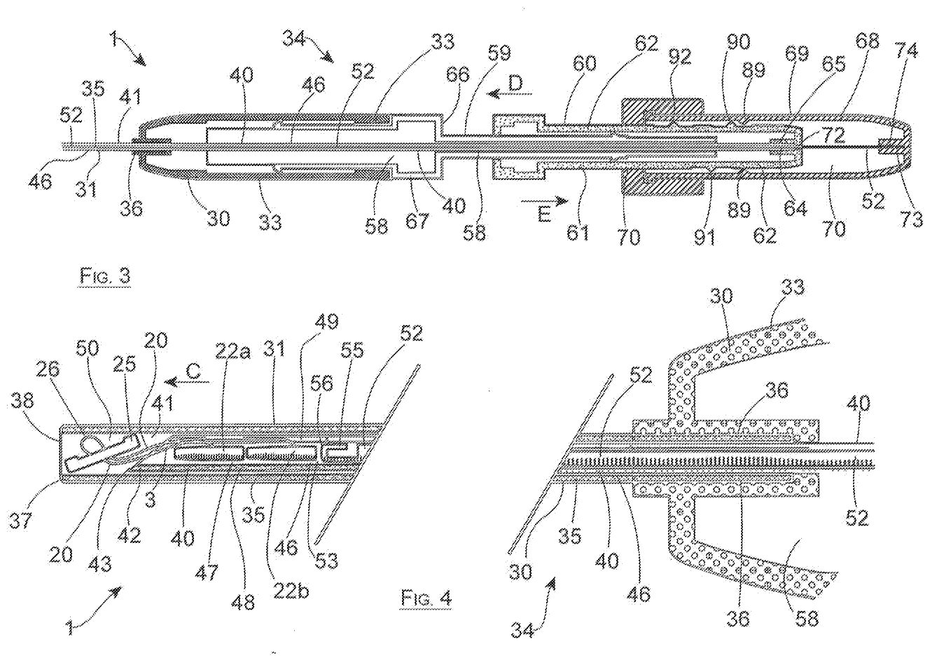

FIG. 2 is a perspective view of another portion of the suturing device of FIG. 1,

FIG. 3 is a cross-sectional side elevational view of the portion of the suturing device of FIG. 1,

FIG. 4 is a cross-sectional side elevational view of the portion of FIG. 2 of the suturing device of FIG. 1,

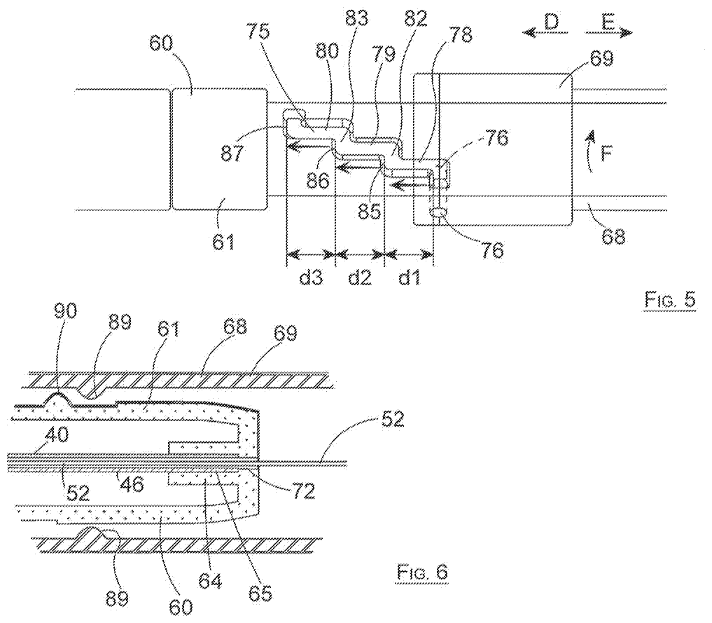

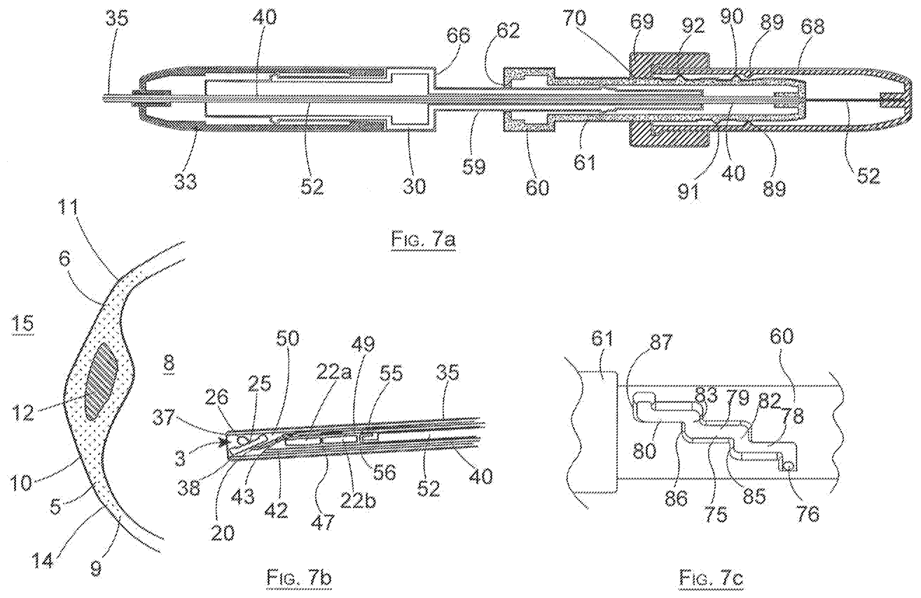

FIG. 5 is a side elevational view of a detail of the suturing device of FIG. 1,

FIG. 6 is an enlarged cross-sectional side elevational view of a detail of the suturing device of FIG. 1,

FIGS. 7a and 7b are cross-sectional side elevational views of portions of the suturing device of FIG. 1 illustrated in the same states, in use,

FIG. 7c is a view of a detail of the suturing device of FIG. 1 illustrated in the state corresponding to the state of the portions of the suturing device illustrated in FIGS. 7a and 7b,

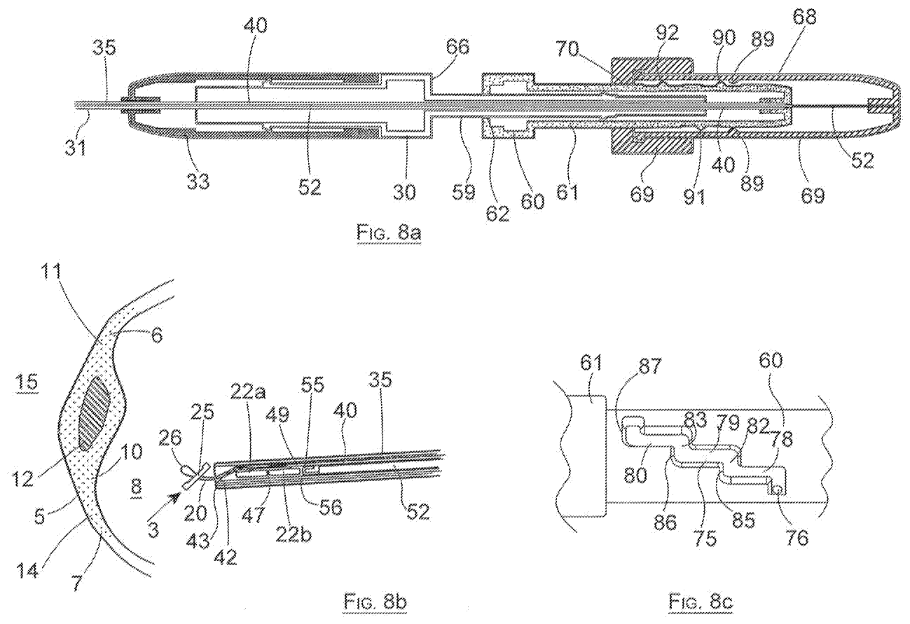

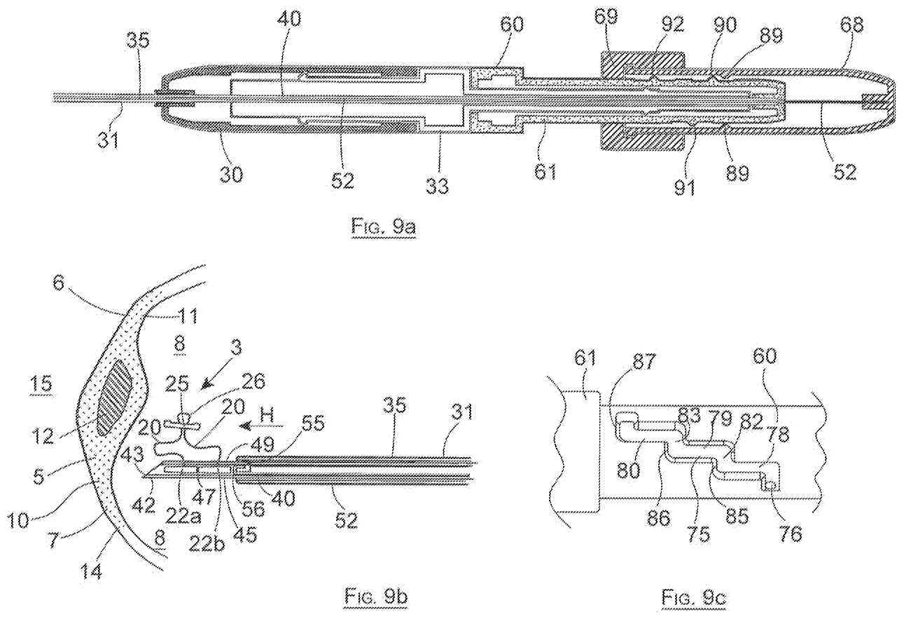

FIGS. 8a and 8b are views of the portions of FIGS. 7a and 7b of the suturing device of FIG. 1 illustrated in a different state in use to that of FIGS. 7a and 7b,

FIG. 8c is a view of the detail of FIG. 7c of the suturing device of FIG. 1 illustrated in a different state in use to that of FIG. 7c,

FIGS. 9a and 9b are views of the portions of FIGS. 7a and 7b of the suturing device of FIG. 1 illustrated in another different state in use to that of FIGS. 7a and 7b,

FIG. 9c is a view of the detail of FIG. 7c of the suturing device of FIG. 1 illustrated in another different state in use to that of FIG. 7c,

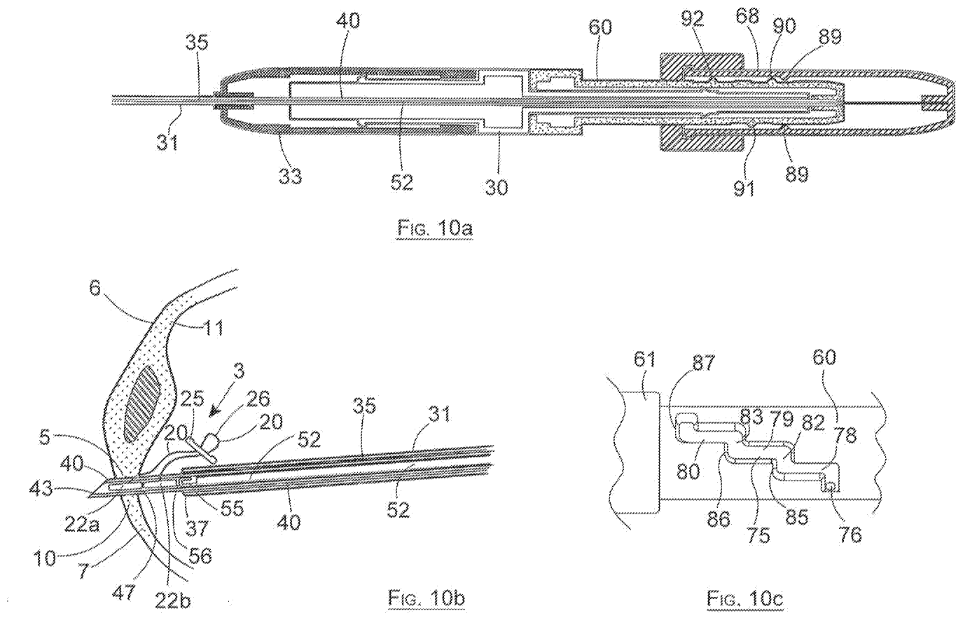

FIGS. 10a and 10b are views of the portions of FIGS. 7a and 7b of the suturing device of FIG. 1 illustrated in a further different state in use to that of FIGS. 7a and 7b,

FIG. 10c is a view of the detail of FIG. 7c of the suturing device of FIG. 1 illustrated in a further different state in use to that of FIG. 7c,

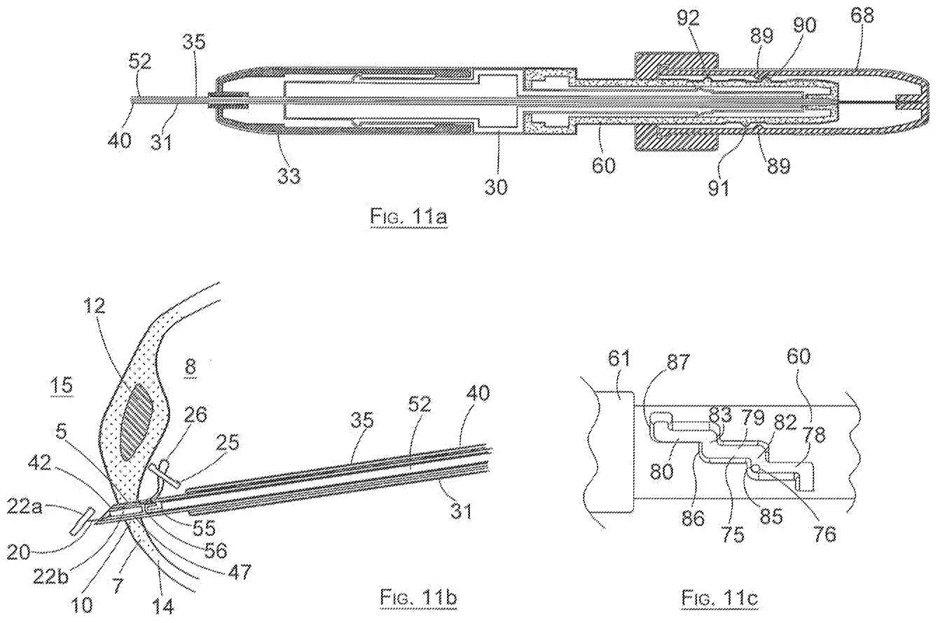

FIGS. 11a and 11b are views of the portions of FIGS. 7a and 7b of the suturing device of FIG. 1 illustrated in a still further different state in use to that of FIGS. 7a and 7b,

FIG. 11c is a view of the detail of FIG. 7c of the suturing device of FIG. 1 illustrated in a still further different state in use to that of FIG. 7c,

FIGS. 12a and 12b are views of the portions of FIGS. 7a and 7b of the suturing device of FIG. 1 illustrated in a still further different state in use to that of FIGS. 7a and 7b,

FIG. 12c is a view of the detail of FIG. 7c of the suturing device of FIG. 1 illustrated in a still further different state in use to that of FIG. 7c,

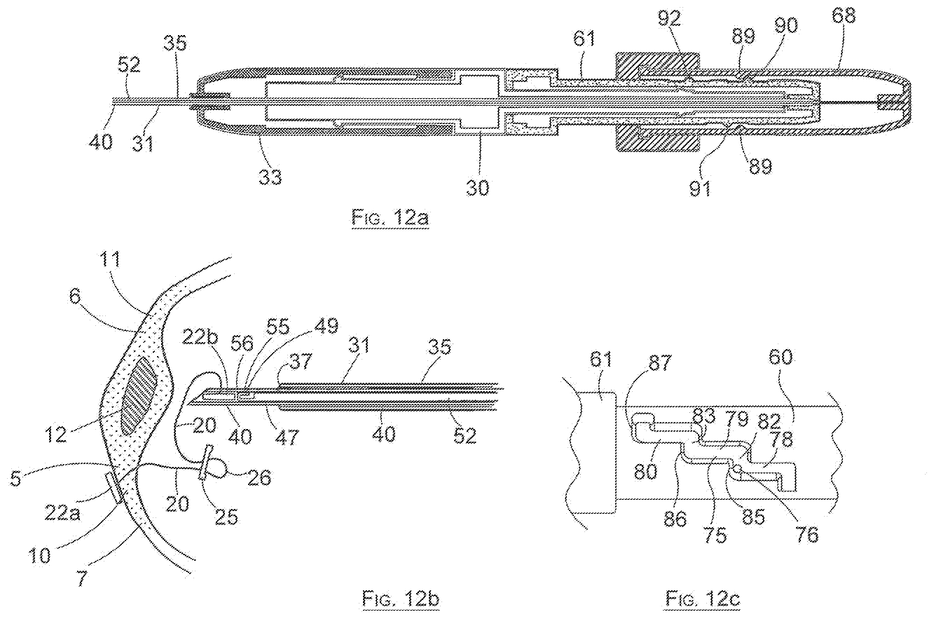

FIGS. 13a and 13b are views of the portions of FIGS. 7a and 7b of the suturing device of FIG. 1 illustrated in a still further different state in use to that of FIGS. 7a and 7b,

FIG. 13c is a view of the detail of FIG. 7c of the suturing device of FIG. 1 illustrated in a still further different state in use to that of FIG. 7c,

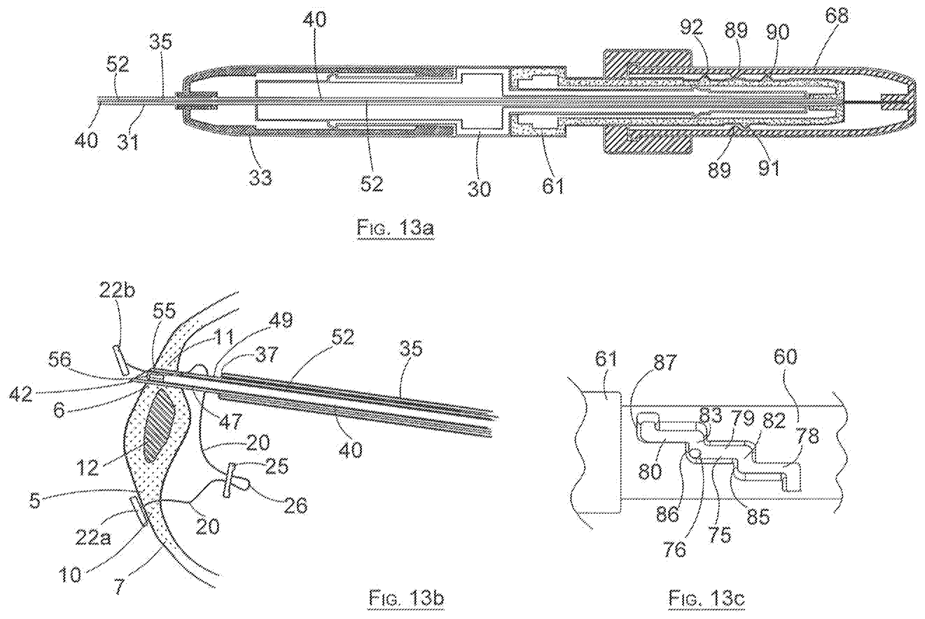

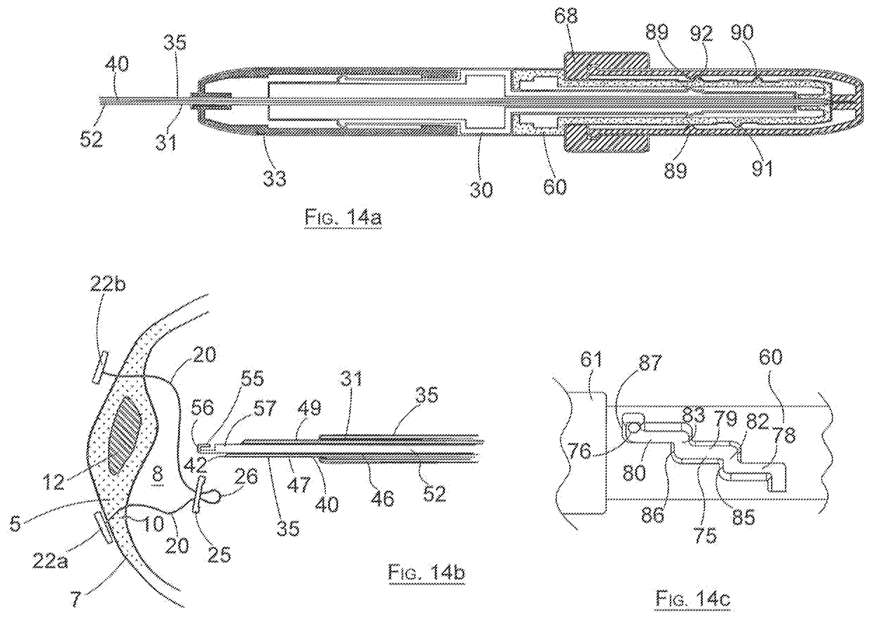

FIGS. 14a and 14b are views of the portions of FIGS. 7a and 7b of the suturing device of FIG. 1 illustrated in a still further different state in use to that of FIGS. 7a and 7b,

FIG. 14c is a view of the detail of FIG. 7c of the suturing device of FIG. 1 illustrated in a still further different state in use to that of FIG. 7c,

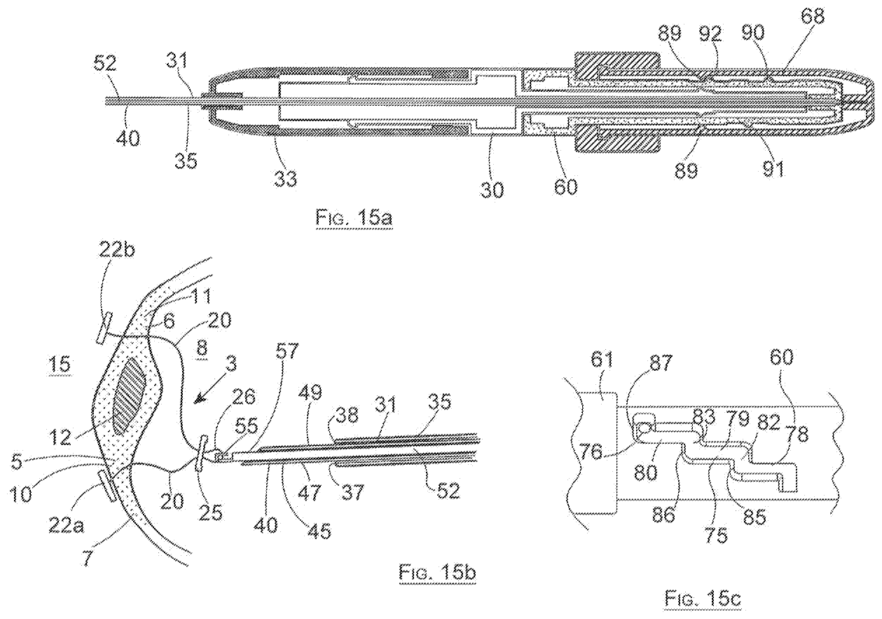

FIGS. 15a and 15b are views of the portions of FIGS. 7a and 7b of the suturing device of FIG. 1 illustrated in a still further different state in use to that of FIGS. 7a and 7b,

FIG. 15c is a view of the detail of FIG. 7c of the suturing device of FIG. 1 illustrated in a still further different state in use to that of FIG. 7c,

FIGS. 16a and 16b are views of the portions of FIGS. 7a and 7b of the suturing device of FIG. 1 illustrated in a still further different state in use to that of FIGS. 7a and 7b,

FIG. 16c is a view of the detail of FIG. 7c of the suturing device of FIG. 1 illustrated in a still further different state in use to that of FIG. 7c,

FIG. 17 is an enlarged perspective view of a suture according to the invention, many times magnified, for use in the suturing device of FIG. 1,

FIG. 18 is a front elevational view of the suture of FIG. 17,

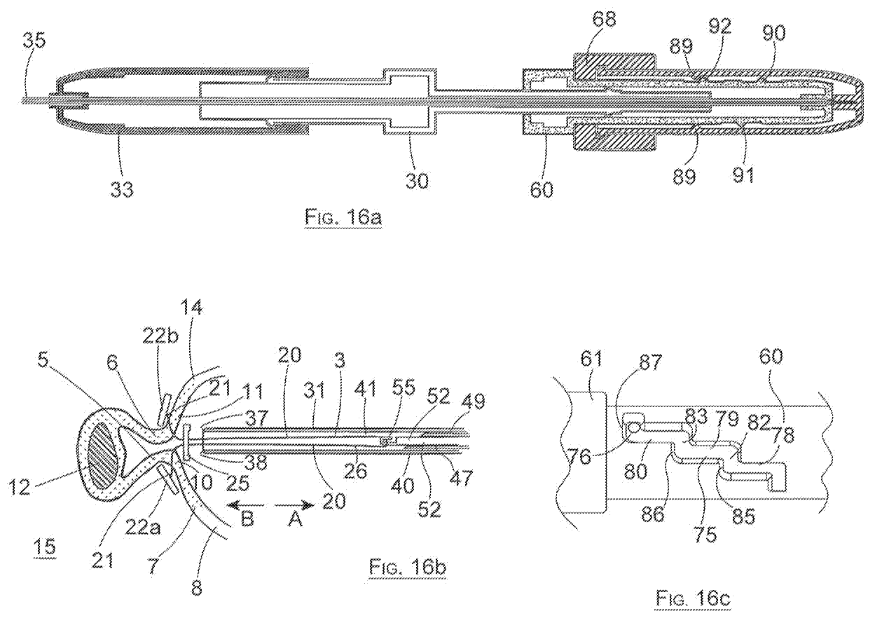

FIG. 19 is an end elevational view of the suture of FIG. 17,

FIG. 20 is a cross-sectional end elevational view of a portion of the suture of FIG. 17 on the line XX-XX of FIG. 18,

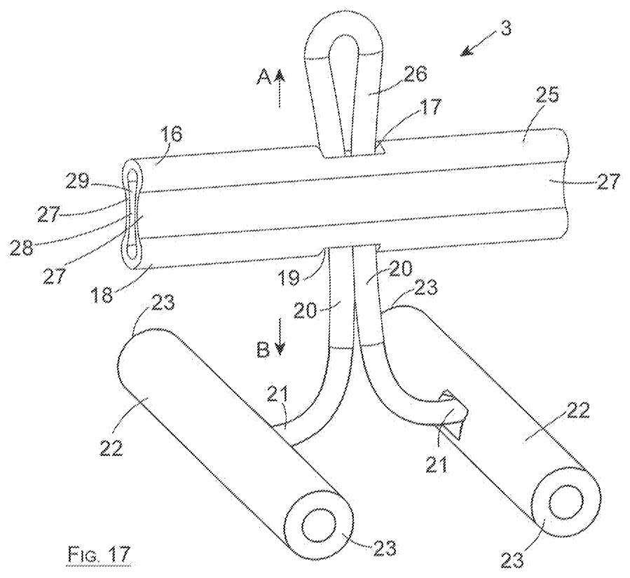

FIG. 21 is a side elevational view of a portion of a suturing device according to another embodiment of the invention,

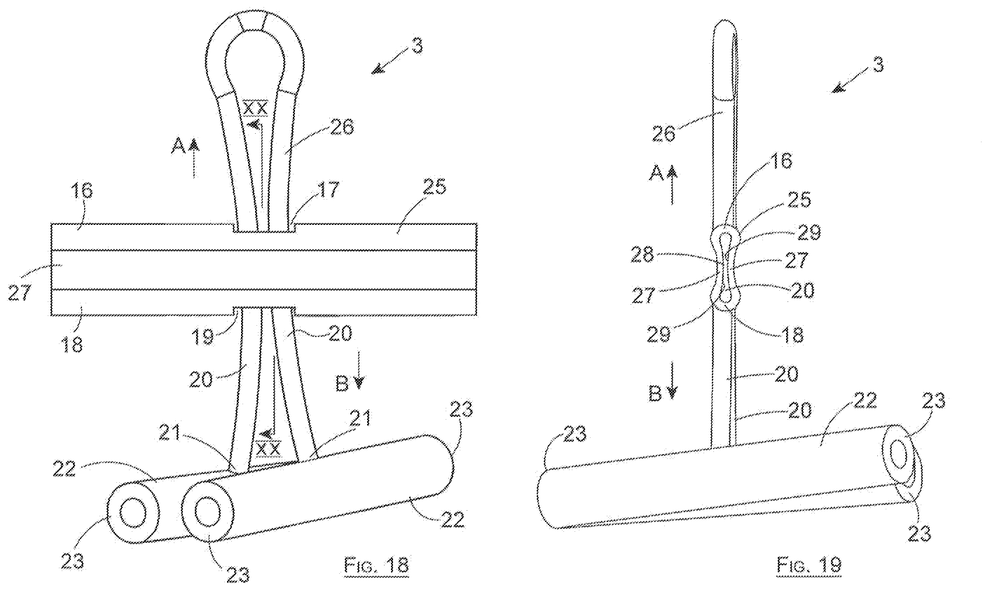

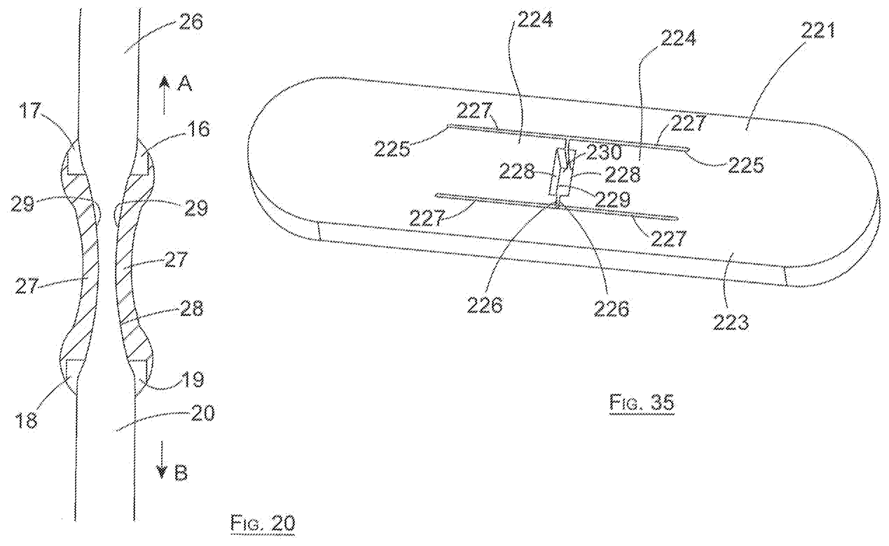

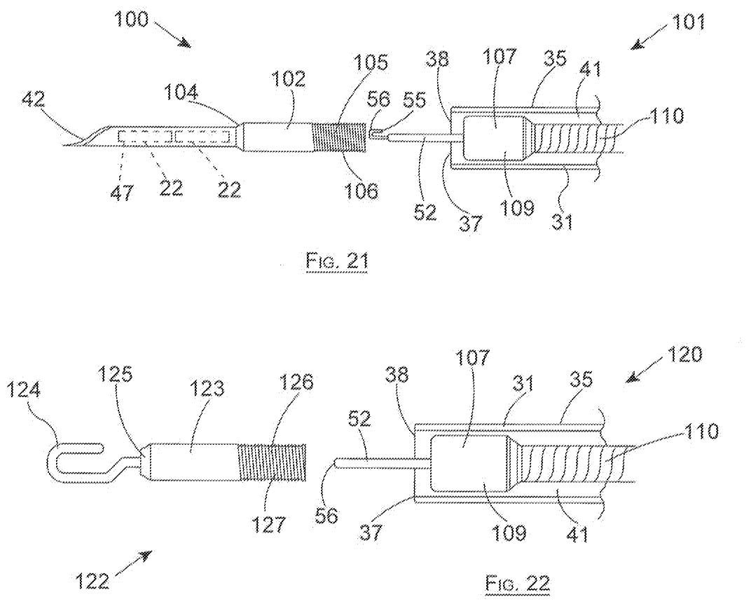

FIG. 22 is a side elevational view of another portion of a suturing device according to a further embodiment of the invention,

FIG. 23 is an enlarged perspective view of a suture, many times magnified, according to another embodiment of the invention,

FIG. 24 is a front elevational view of the suture of FIG. 23,

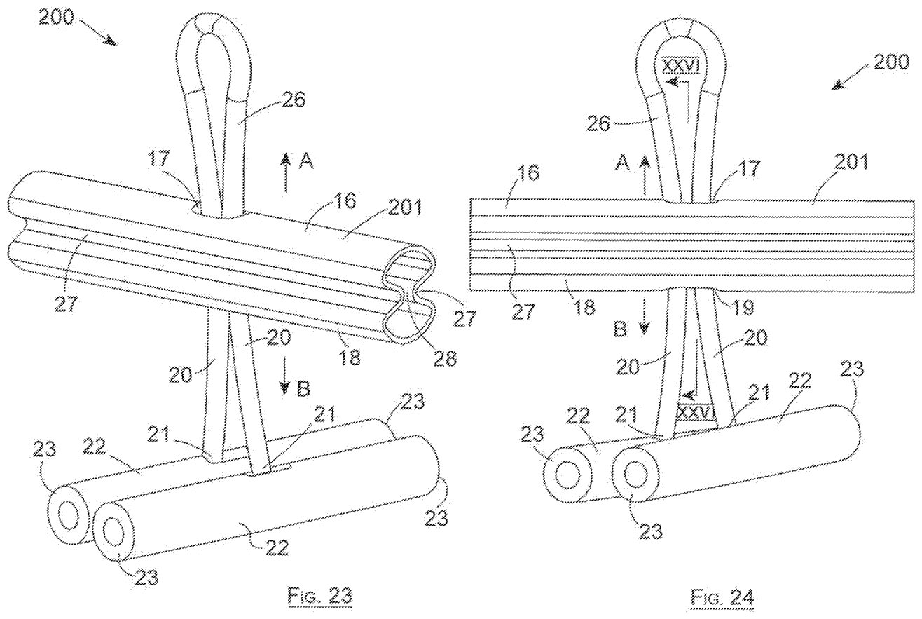

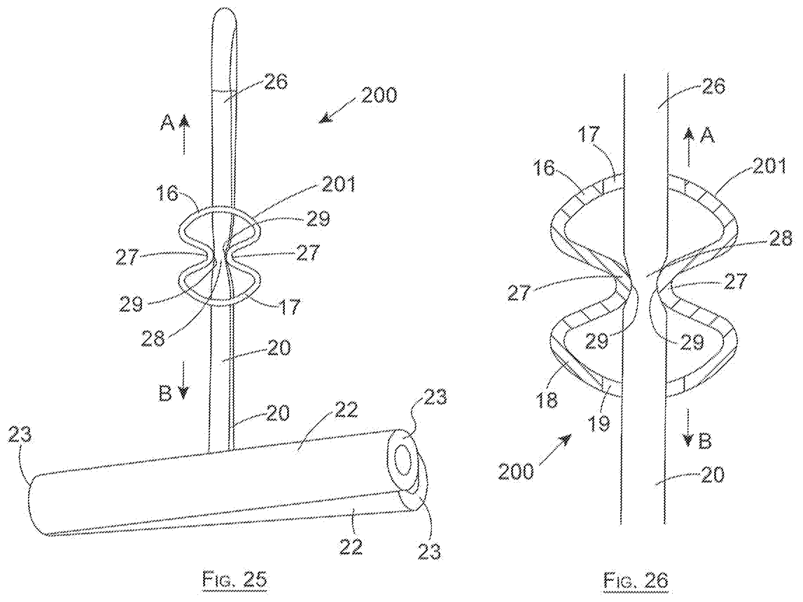

FIG. 25 is an end elevational view of the suture of FIG. 23,

FIG. 26 is a transverse cross-sectional end elevational view of the suture of FIG. 23 on the line XXVI-XXVI of FIG. 24,

FIG. 27 is an enlarged perspective view of a suture, many times magnified, according to another embodiment of the invention,

FIG. 28 is a front elevational view of the suture of FIG. 27,

FIG. 29 is an end elevational view of the suture of FIG. 27,

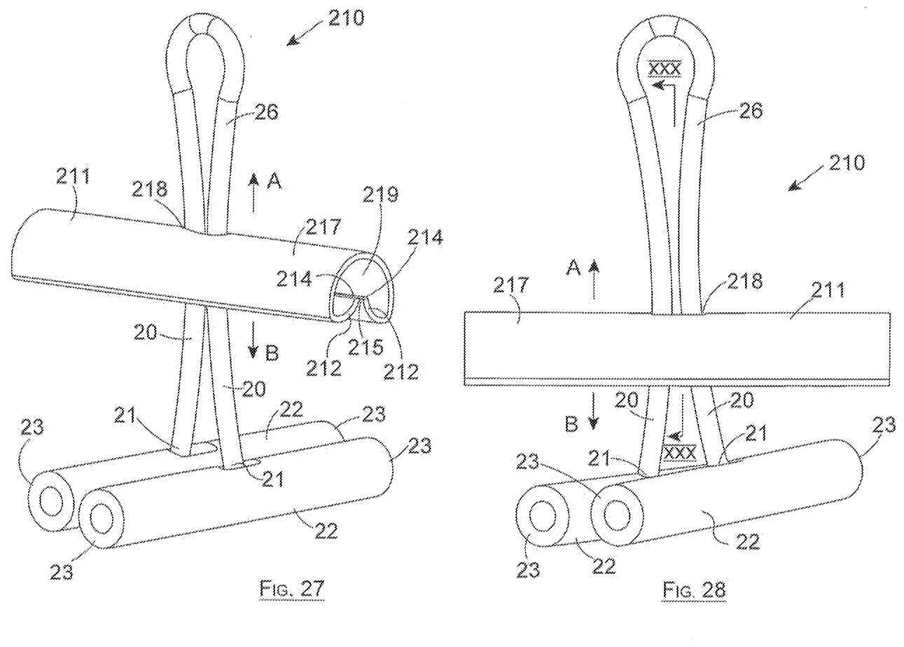

FIG. 30 is a transverse cross-sectional end elevational view of the suture of FIG. 27 on the line XXX-XXX of FIG. 28,

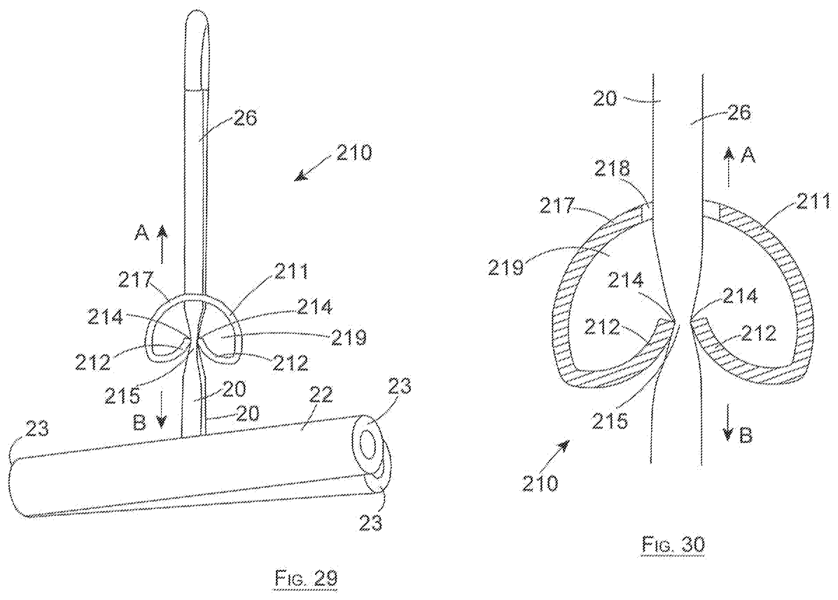

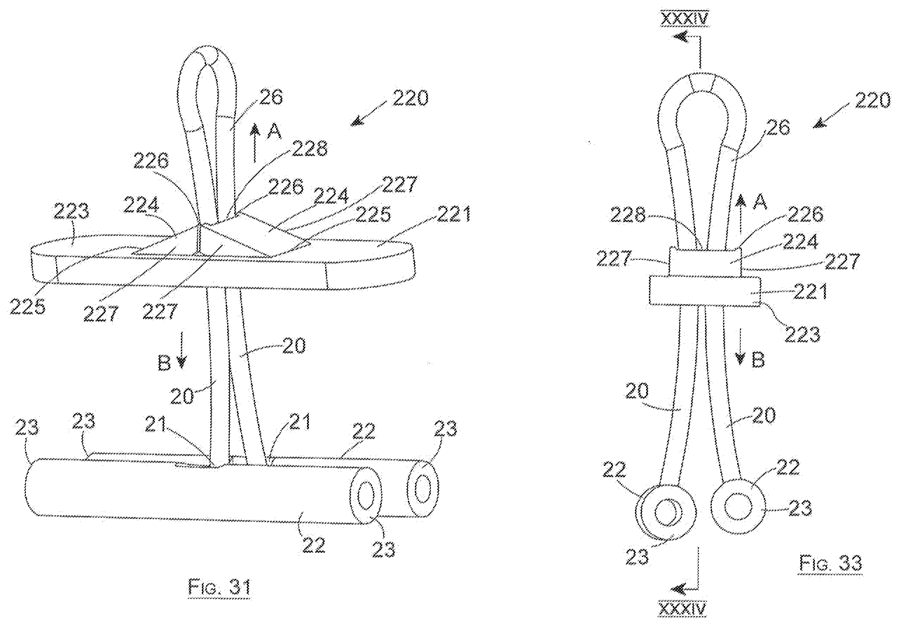

FIG. 31 is an enlarged perspective view of a suture, many times magnified, according to another embodiment of the invention,

FIG. 32 is a front elevational view of the suture of FIG. 31,

FIG. 33 is an end elevational view of the suture of FIG. 31,

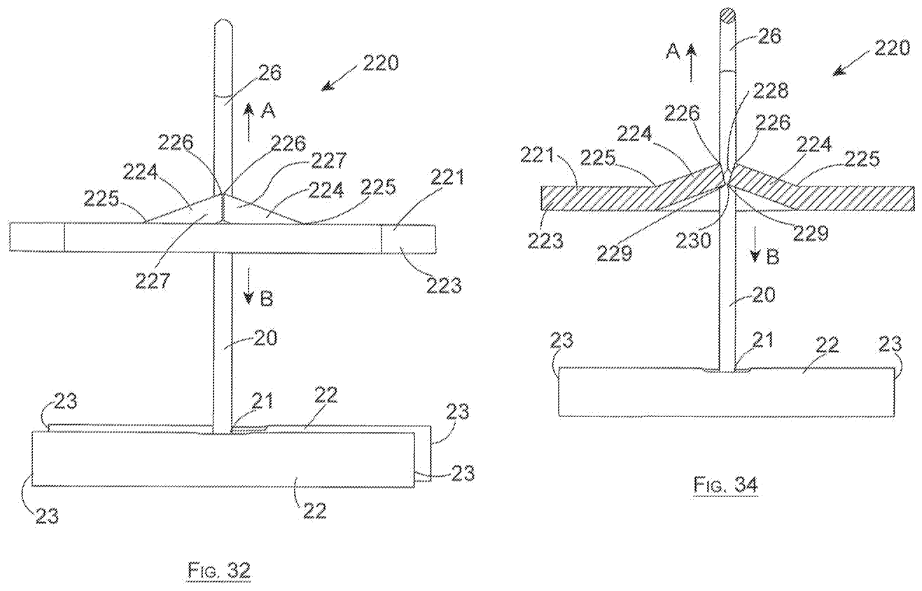

FIG. 34 is a transverse cross-sectional end elevational view of the suture of FIG. 31 on the line XXXIV-XXXIV of FIG. 33,

FIG. 35 is a perspective view of a part of the suture of FIG. 31,

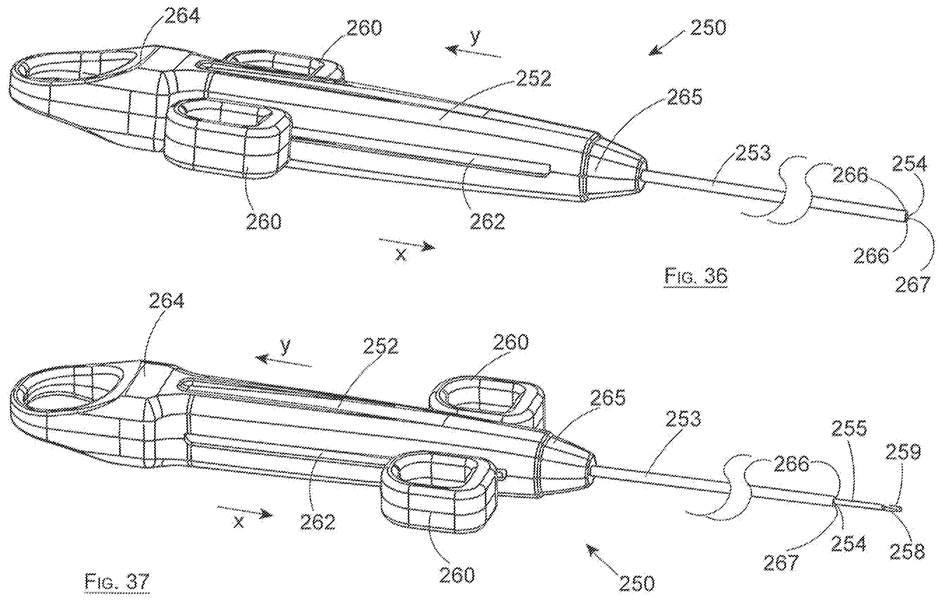

FIG. 36 is a perspective view of a suture securing instrument also according to the invention, and

FIG. 37 is another perspective view of the suture securing instrument of FIG. 36 illustrated in a different state to that of FIG. 36.

Referring to the drawings, and initially to FIGS. 1 to 16 thereof, there is illustrated a suturing device according to the invention indicated generally by the reference numeral 1 for suturing two parts of a human or animal body of a subject together with a suture also according to the invention and indicated generally by the reference numeral 3. The suture 3 is described below with reference to FIGS. 17 to 20. In this embodiment of the invention the suturing device 1 is described for suturing two parts, namely, a first part and a second part of the human body of the subject, in this case, first and second parts 5 and 6, respectively, of a colon wall 7 of a colon 8 on respective opposite sides 10 and 11 of a polyp 12 in the colon wall 7. The first and second parts 5 and 6 of the colon wall 7 are sutured together by the suture 3 in order to evert the polyp 12 to an outer side 14 of the colon wall 7, so that the polyp 12 can be subsequently severed from the colon wall 7 in the abdominal cavity 15 of the subject.

Before describing the suturing device 1, the suture 3 will first be described with reference to FIGS. 17 to 20. The suture 3 terminates at its respective opposite ends 21 in respective anchor elements, in this embodiment of the invention provided by transverse members 22 comprising bars of circular transverse cross-section. Each end 21 of the suture 3 is secured in the corresponding transverse member 22 substantially midway between the respective opposite ends 23 of the corresponding transverse member 22.

An adjustment element 25 is engageable with a loop 26 of the suture 3 extending between the transverse members 22. The loop 26 defines a pair of legs 20 of the suture 3 terminating in the respective transverse member 22. The adjustment element 25 is urgeable along the loop 26 in the directions of the arrows A and B for adjusting the effective length of the suture 3 between the transverse members 22. By urging the adjusting element 25 along the loop 26 towards the transverse members 22 in the direction of the arrow B, the effective length of the suture 3 is shortened, for in turn urging the first and second portions 5 and 6 of the colon wall 7 together, when the legs 20 of the suture 3 have been inserted through the colon wall 7 adjacent the respective first and second parts 5 and 6 with the transverse members 22 abutting the outer side 14 of the colon wall 7, see FIG. 16b, as will be described below.

The adjustable element 25 comprises a retainer for engaging the legs 20 of the loop 26, and for retaining the adjustment element 25 at a selectable location along the loop 26 with the effective length of the suture 3 between the transverse members 22 at a desired selected length, for in turn retaining the first and second parts 5 and 6 of the colon wall 7 together. The retainer comprises a pair of spaced apart abutment members 27 defining abutment faces 29 facing each other and forming a suture accommodating gap 28 therebetween for accommodating the legs 20 of the loop 26 therethrough. A first cross-member 16 extends between and joins the abutment members 27 at one end of the suture accommodating gap 28. A suture accommodating opening 17 in the first cross-member 16 accommodates the loop 26 of the suture 3 therethrough from the suture accommodating gap 28. A second cross-member 18 extends between and joins the abutment elements 27 at the other end of the suture accommodating gap 28. A suture accommodating opening 19 extending through the second cross-member 18 accommodates the legs 20 of the loop 26 of the suture 3 into the suture accommodating gap 28.

The first and second cross-members 16 and 18 are of arcuate shape, and each cross-member 16 and 18 when viewed through the suture accommodating gap 28 is of transverse concave arcuate cross-section. The cross-members 16 and 18 are configured to retain the position of the abutment members 27 substantially fixed relative to each other, in other words to prevent outward movement of the abutment members 27 relative to each other, in order to tightly engage the suture 3 therebetween in the suture accommodating gap 28 with a transversely directed engagement force of predefined value.

The predefined value of the engagement force acting on the legs 20 of the suture 3 by the abutment members 27 is such as to prevent movement of the legs 20 of the loop 26 of the suture 3 through the suture accommodating gap 28 until the pulling force acting between the adjustment element 25 and the corresponding transverse members 22 exceeds a predefined value. The predefined pulling force value is greater than the force induced in the legs 20 of the suture 3 between the adjustment element 25 and the respective transverse members 22 by the effect of the parts 5 and 6 of the colon wall 7 acting on the transverse members 22 when the parts 5 and 6 have been pulled tightly together by the suture 3. Thereby the suture 3 retains the portions 5 and 6 of the colon wall 7 tightly adjacent each other, for in turn everting the polyp outwardly on the outer side 14 of the colon wall 7.

The predefined engagement force with which the abutment members 27 engage the legs 20 of the loop 26 is such as to permit the loop 26 to be pulled through the suture accommodating gap 28 in the direction of the arrow A, for in turn urging the adjustment element 25 along the loop 26 in the direction of the arrow B to the selected location on the loop 26 for retaining the portions 5 and 6 of the colon wall 7 together once the pulling force exerted on the loop 26 is such that the pulling forces in the respective legs 20 of the loop 26 relative to the adjustment element 25 exceeds the predefined pulling force value. Thus, the adjustment element 25 will be selected so that the abutment members 27 engage the legs 20 of the loop 26 of the suture 3 with a predefined engagement force value to prevent movement of the legs 20 of the suture 3 through the adjustment element 25 until the pulling force in the legs 20 of the suture 3 between the adjustment element 25 and the corresponding transverse members 22 exceeds the predefined pulling force value.

The predefined pulling force value and in turn the predefined engagement force value will be dependent on the force which will be required to urge the two parts of an organ or other tissue in the human or animal body together and to retain the two parts of the tissue together by the suture 3.

In this embodiment of the invention the adjustment element 25 is formed from a tubular member of stainless steel material of circular transverse cross-section, and is subsequently deformed to be of the desired cross-section, which in this case is of transverse cross-sectional shape substantially similar to a figure of eight or an hourglass. The first arcuate cross-member 16 forms the upper arcuate portion of the figure of eight or the hourglass, while the second cross-member 18 forms the lower arcuate portion of the figure of eight or the hourglass. The abutment members 27 essentially form the sides of the narrowing central portion of the figure of eight or the hourglass.

The suturing device 1 as will be described below is configured for urging the suture 3 through the first part 5 of the colon 8 for depositing one of the transverse members 22 on the outer side 14 of the colon wall 7 adjacent the first part 5 externally of the colon 8, and then for urging the suture 3 through the second part 6 of the colon wall 7 and depositing the other one of the transverse members 22 on the outer side 14 of the colon wall 7 adjacent the second part 6 externally of the colon 8. The loop 26 of the suture 3 is then pulled through the adjustment element 25 by the suturing device 1 for progressively shortening the effective length of the suture between the transverse members 22, for in turn urging the first and second parts 5 and 6 of the colon 8 together to thereby everting the polyp 12 onto the outer side 14 of the colon wall 7 as illustrated in FIG. 16b.

Turning now to the suturing device 1, and referring in particular to FIGS. 1 to 6, and 7 to 16, the suturing device 1 comprises a cannula 40 for accommodating the suture 3 therein, and for manipulating the suture 3 in the colon 8, for in turn suturing the two parts 5 and 6 together, and an actuator 34 for operating the cannula 40 for suturing the two parts 5 and 6 of the colon 8 together, as will also be described in detail below.

The actuator 34 comprises a carrier which in this embodiment of the invention comprises a first carrier element 30 and a second carrier element 31 extending distally from the first carrier element 30. The first carrier element 30 is of a plastics material and forms a handle 33 of the suturing device 1 for gripping and holding the suturing device 1. The second carrier element 31 comprises an elongated tubular shield 35 of circular transverse cross-section of a resiliently flexible material of the construction of a Bowden cable, and in this case, of stainless steel material. The tubular shield extends distally from the first carrier element 30.

Before describing the first carrier element 30, the tubular shield 35 forming the second carrier element 31 and the components therein will first be described.

The tubular shield 35 is rigidly secured in a bore 36 of the first carrier element 30 and extends distally therefrom, terminating in a distal end 37 which forms an abutment element, namely, an abutment face 38 for engaging the adjustment element 25 of the suture 3 as the loop 26 is being urged therethrough as will be described below. An elongated cannula accommodating bore 41 of circular transverse cross-section extends longitudinally through the tubular shield 35 for accommodating the cannula 40 therein.

The cannula 40, in this embodiment of the invention, comprises an elongated cannula of circular transverse cross-section located in the cannula accommodating bore 41 of the tubular shield 35 and slideable longitudinally therein. The cannula 40 extends proximally through the cannula accommodating bore 41 into the first carrier element 30 of the actuator 34, so that the cannula 40 is urgeable through the cannula accommodating bore 41 of the tubular shield 35 by the actuator 34 as will be described below. The cannula 40 terminates at its distal end 42 in a rigid piercing tip 43 for piercing through the first and second parts 5 and 6 of the colon wall 7. The portion of the cannula 40 extending proximally from the rigid piercing tip 43 into the first carrier element 30 of the actuator 34 comprises a resiliently flexible tubular material, so that it is resiliently flexible and bendable with the tubular shield 35. In this embodiment of the invention the rigid piercing tip 43 and the proximally extending portion of the cannula 40 are of stainless steel material. The cannula 40 is slideable in the cannula accommodating bore 41, and is urgeable by the actuator 34 as will be described below in a distal direction, namely, in the direction of the arrow C from a withdrawn state illustrated in FIG. 4 located within the cannula accommodating bore 41 of the tubular shield 35 to an extended state illustrated in FIGS. 9b and 10b with a distal portion 45 of the cannula 40 extending outwardly and distally from the abutment face 38 of the tubular shield 35 for sequentially piercing the first and second parts 5 and 6 of the colon wall 7.

An elongated bore 46 of circular transverse cross-section extends through the cannula 40, and a distal portion 48 of the cannula bore 46 defines a suture accommodating chamber 47 for accommodating the suture 3 therein. The suture 3 is located in the suture accommodating chamber 47 with the transverse members 22 thereof sequentially arranged in the suture accommodating chamber 47, so that the transverse members 22 of the suture 3 are urgeable sequentially from the suture accommodating chamber 47 through the distal end 42 of the cannula 40 for sequentially depositing the transverse members 22 on the outer side 14 of the colon wall 7 adjacent the first and second parts 5 and 6 thereof. The diameter of the transverse members 22 of the suture 3 and the diameter of the cannula bore 46 in the suture accommodating chamber 47 is such that the transverse members 22 are slideable in and through the suture accommodating chamber 47 for discharge therefrom through the distal end 42 of the cannula 40, as will be described below.

An elongated longitudinally extending suture accommodating slot 49 extends into the cannula 40 from the distal end 42 thereof for accommodating the suture 3 therefrom.

The cannula 40 when in the withdrawn state illustrated in FIG. 4 defines with the tubular shield 35 an adjustment element accommodating chamber 50 in the cannula accommodating bore 41 between the distal end 37 of the tubular shield 35 and the rigid piercing tip 43 of the cannula 40 for accommodating the adjustment element 25 of the suture 3 therein prior to urging of the cannula 40 distally from the withdrawn state in the tubular shield 35.

An urging element comprising a push rod 52 extends longitudinally through the cannula bore 46 from the actuator 34, as will be described below, and terminates adjacent the proximal end 53 of the suture accommodating chamber 47. In this embodiment of the invention the push rod 52 comprises an elongated wire, which is resiliently flexible to flex with and bend with the cannula 40 and the tubular shield 35, and is of stainless steel material. The push rod 52 as will be described below is urgeable distally through the cannula bore 46, and in turn through the suture accommodating chamber 47 for sequentially urging the transverse members 22 from the suture accommodating chamber 47 through the distal end 42 of the cannula 40.

In this embodiment of the invention the push rod 52 terminates in an engagement element provided by a hook 55 for engaging the loop 26 of the suture 3 for urging the loop 26 through the adjustment element 25 in the direction of the arrow A as will be described below, for in turn urging the first and second parts 5 and 6 of the colon wall 7 together. The wire of the push rod 52 is of circular transverse cross-section and is of diameter such that it is slideable longitudinally in the cannula bore 46. The hook 55 defines a similar circular transverse cross-section to that of the push rod 52. In this embodiment of the invention the hook 55 forms a part of the urging element.

The hook 55 terminates in an engagement end 56, which when the push rod 52 is in a withdrawn state illustrated in FIG. 4, defines with the distal portion 48 of the cannula 40 the suture accommodating chamber 47 in the cannula bore 46. The engagement end 56 of the hook 55 is configured for engaging the proximal most one of the transverse members 22 of the suture 3 for sequentially urging the transverse members 22 of the suture 3 through the distal end 42 of the cannula 40.

In this embodiment of the invention the push rod 52 is urgeable distally by the actuator 34, as will be described below, through a first predefined distance from the withdrawn state for urging the distal most one of the transverse members, namely, the first transverse member 22a of the suture 3 from the suture accommodating chamber 47, and in turn through the distal end 42 of the cannula 40. The push rod 52 is urgeable distally by the actuator 34 through a second predefined distance after the first predefined distance for urging the proximal most one of the transverse members, namely, the second transverse member 22b of the suture 3 from the suture accommodating chamber 47 through the distal end 42 of the cannula 40.

The push rod 52 is also urgeable from the withdrawn state to an extended state illustrated in FIGS. 2, 14b and 15b with the hook 55 and a distal portion 57 of the push rod 52 extending distally outwardly from the piercing tip 43 of the cannula 40 for engaging the loop 26 of the suture 3 with the hook 55. In this embodiment of the invention the push rod 52 is urgeable to the extended state through a fifth predefined distance after it has been urged through the second predefined distance.

The tubular shield 35, the cannula 40 and the push rod 52 may be of any suitable length, and the length thereof to some extent, will be dictated by the location in the body of the subject of the site at which the suturing is to be carried out.

Turning now to the actuator 34, the first carrier element 30 which forms the handle 33, also comprises a carrier spigot 59 of a plastics material extending proximally from the handle 33. The handle 33 and the carrier spigot 59 define a hollow interior region 58 for accommodating the cannula 40 therethrough. A first operating element 60 comprising a first body member 61 of a plastics material and having a first bore 62 extending into the first body member 61 is slideably mounted on the carrier spigot 59 with the carrier spigot 59 slideably engaging the first bore 62 so that the first operating element 60 is slideable longitudinally on the first carrier spigot 59 in a first direction, namely, in the direction of the arrow D, and a second direction, namely, in the direction of the arrow E opposite to the first direction.

The cannula 40 terminates at its proximal end 64 in a first engagement bore 65 in the first body member 61 of the first operating element 60 and is secured therein so that the cannula 40 is urgeable through the cannula accommodating bore 41 of the tubular shield 35 by the first operating element 60 as will be described below.

A second operating element 68 comprises a second body member 69, having a second bore 70 extending into the second body member 69. The second bore 70 of the second body member 69 is slideably mounted on the first body member 61 of the first operating element 60, so that the second operating element 68 is longitudinally slideable on the first operating element 60 in first and second directions, namely, in the directions of the arrows D and E, respectively. The push rod 52 extends from the proximal end 64 of the cannula 40 through a push rod accommodating bore 72 in the first body member 61 of the first operating element 60, and terminates at its proximal end 73 in a second engagement bore 74 in the second body member 69 of the second operating element 68, and is secured therein so that the push rod 52 is urgeable through the cannula bore 46 by the second operating element 68.

The first operating element 60 is keyed relative to the carrier spigot 59 by a key and keyway (not shown) for preventing rotational movement of the first operating element 60 relative to the carrier spigot 59. However, the first operating element 60 is slideable longitudinally on the carrier spigot 59 in the direction of the arrow D from a first state illustrated in FIGS. 3 and 7a to a second state illustrated in FIGS. 9a to 13b abutting an end stop 66 formed by the proximal end 67 of the handle 33, for urging the cannula 40 in the direction of the arrow H from the withdrawn state within the cannula accommodating bore 41 of the tubular shield 35 illustrated in FIG. 4 to the extended state illustrated in FIGS. 9b to 13b with the distal portion 45 of the cannula 40 extending outwardly distally from the abutment face 38 of the tubular shield 35 for sequentially piercing the first and second parts 5 and 6 of the colon wall 7 for inserting the suture 3 in the first and second parts 5 and 6 thereof, see FIGS. 10b and 13b. As the cannula 40 is urged from the withdrawn state to the extended state, the adjustment element 25 of the suture 3 is urged by the cannula 40 from the suture accommodating chamber 50 through the distal end 37 of the tubular shield 35.

Turning now to the second operating element 68, the second operating element 68 is urgeable in the first direction, namely, in the direction of the arrow D relative to the first operating element 60 from a first state illustrated in FIGS. 3, 5 and FIG. 7a through a third predefined distance, namely, the distance d₁, see FIG. 5, to a second state illustrated in FIG. 11a, for urging the push rod 52 through the first predefined distance for in turn urging the distal most one of the transverse members 22 of the suture 3, namely, the first transverse member 22a of the suture 3 through the distal end 42 of the cannula 40. The second operating element 68 is urgeable from the second state to a third state illustrated in FIG. 13b in the first direction, namely, in the direction of the arrow D relative to the first operating element 60 through a fourth predefined distance d₂, see FIG. 5, for urging the push rod 52 through the second predefined distance for in turn urging the proximal most one of the transverse members 22 of the suture 3, namely, the second transverse member 22b of the suture 3 through the distal end 42 of the cannula 40.

The second operating element 68 is urgeable relative to the first operating element 60 in the first direction, namely, in the direction of the arrow D from the third state to a fourth state illustrated in FIG. 14a through a sixth predefined distance d₃, see FIG. 5, for in turn urging the push rod 52 distally through the fifth predefined distance to the extended state thereof for in turn urging the engagement hook 55, and in turn the distal portion 57 of the push rod 52 through the distal end 42 of the cannula 40 for engaging the hook 55 in the loop 26 of the suture 3 as illustrated in FIGS. 14b and 15b.

In this embodiment of the invention a guide means is provided for guiding the second operating element 68 relative to the first operating element 60 through the third, fourth and sixth predefined distances d₁, d₂ and d₃, respectively, so that on each movement of the second operating element 68 through the respective third, fourth and sixth predefined distances, the second operating element 68 is constrained to move only through the corresponding third, fourth and sixth predefined distances. In this embodiment of the invention the guide means comprises a camming groove 75 which is formed in the first operating element 60, and a cam follower 76 which extends from the second operating element 68 into the bore 70 of the second operating element 68 and in turn into the camming groove 75, see FIGS. 1 and 5, and FIGS. 7c to 16c.

The camming groove 75 comprises three longitudinally extending camming groove segments, namely, a first longitudinally extending camming groove segment 78, a second longitudinally extending camming groove segment 79 and a third longitudinally extending camming groove segment 80. The first and second camming groove segments 78 and 79 are joined by a first circumferentially extending camming groove segment 82, while the second and third longitudinally extending camming groove segments 79 and 80 are joined by a second circumferentially extending camming groove segment 83. Both the first and second circumferentially extending camming groove segments 82 and 83 extend transversely of the first, second and third longitudinally extending camming grooves 78, 79 and 80. The first, second and third longitudinally extending camming groove segments 78, 79 and 80 are of lengths which define and determine the third, fourth and sixth predefined distances d₁, d₂ and d₃, respectively.

Distal ends 85, 86 and 87 of the first, second and third longitudinally extending camming groove segments 78, 79 and 80, respectively, act as first, second and third stop elements 85, 86 and 87, respectively, for limiting the movement of the cam follower 76 in the camming groove 75, so that after the second operating element 68 has been urged through the third predefined distance d₁ with the cam follower 76 engaging the first stop element 85, the second operating element 68 cannot be urged through the fourth predefined distance d₂ until the second operating element 68 has been twisted relative to the first operating element 60 for urging the cam follower 76 through the first circumferentially extending camming groove segment 82 into alignment with the second camming groove segment 79. This, thus, ensures that only the first transverse member 22a of the suture 3 is dispensed from the suture accommodating chamber 47 in response to the second operating element 68 being urged through the third predefined distance d₁. There is no danger of the second transverse member 22b of the suture 3 being inadvertently discharged from the suture accommodating chamber 47, until the second operating element 68 has been twisted relative to the first operating element 60 for aligning the cam follower 76 with the second longitudinally extending camming groove segment 79.

Additionally, once the cam follower 76 has reached the second stop element 86 at the end of the second longitudinally extending camming groove segment 79, the second operating element 68 cannot be urged further in the first direction, namely, in the direction of the arrow D relative to the first operating element 60, until the second operating element 68 has been twisted relative to the first operating element 60, in order to align the cam follower 76 with the third longitudinally extending camming groove segment 80.

In this embodiment of the invention the first and third predefined distances are equal to each other, the second and fourth predefined distances are equal to each other, and the fifth and sixth predefined distances are also equal to each other. Additionally, the first, second, third and fourth predefined distances are also equal to each other.

Additionally, in this embodiment of the invention complementary projections, namely, a pair of first projections 89 extend radially from the second operating element 68 into the second bore 70 thereof for sequentially engaging second, third and fourth projections 90, 91 and 92 extending radially outwardly from the first operating element 60 as the cam follower 76 of the second operating element 68 reaches the end of each of the first, second and third longitudinally extending camming groove segments 78, 79 and 80 to indicate to a user that the second operating element 68 has moved through the respective third, fourth and sixth predefined distances.

In use, with the suture 3 located in the suture accommodating chamber 47 at the distal end 48 of the cannula 40, with the transverse members 22 of the suture 3 located in the suture accommodating chamber 47, and with the cannula 40 in the withdrawn state within the cannula accommodating bore 41 of the tubular shield 35 and the adjustment element 25 in the adjustment element accommodating chamber 50, and with the push rod 52 in the withdrawn state within the cannula bore 46, the suturing device 1 is ready for use. With the cannula 40 in the withdrawn state and the push rod 52 in the withdrawn state, the first operating element 60 is in the first state thereof on the carrier spigot 59, and the second operating element 68 is in the first state thereof on the first operating element 60.

Assuming that it is desired to suture the first and second parts 5 and 6 of the colon wall 7 together, in order to evert the polyp 12 from the internal side of the colon wall 7 to the outer side 14 of the colon wall 7 for subsequent severing thereof, a colonoscope (not shown) is initially entered rectally into the colon 8. The tubular shield 35 of the suturing device 1 is entered into the colon 8 through a working channel in the colonoscope. Once the tubular shield 35 has been entered into the colon 8 through the rectum, and the distal end 37 of the tubular shield 35 is located adjacent the first part 5 of the colon 8, the first operating element 60 is urged from the first state in the first direction, namely, in the direction of the Arrow D into the second state abutting the end stop 66 defined by the end 67 of the handle 33, for in turn urging the cannula 40 from the withdrawn state into the extended state with the distal end 42 of the cannula 40 extending distally outwardly through the distal end 37 of the tubular shield 35, see FIGS. 7a to 7c, to 9a to 9c. As the cannula 40 is being urged distally through the tubular shield 35 through the adjustment element accommodating chamber 50, the adjustment element 25 of the suture 3 is discharged through the distal end 37 of the tubular shield 35 into the colon 8, see FIG. 8b. The piercing tip 43 is then urged through the first part 5 of the colon wall 7 by urging the tubular shield 35 further into the colon 8, see FIG. 10b.

Once the piercing tip 43 of the cannula 40 has pierced through the first part 5 of the colon wall 7, the second operating element 68 is urged relative to the first operating element 60 in the first direction of the arrow D from the first state thereof through the third predefined distance d₁ to the second state thereof, and the cam follower 76 travels along the first longitudinally extending camming groove segment 78 to the first stop element 85 thereof, see FIGS. 11a to 11c. At this stage the push rod 52 has been urged through the first predefined distance and the first transverse member 22a located in the suture accommodating chamber 47 has been urged through the distal end 42 of the cannula 40 to be located on the outer side 14 of the colon 8 with the suture extending from the first transverse member 22a through the first part 5 of the colon wall 7 into the colon 8, see FIG. 11b. The piercing tip 43 of the cannula 40 is then withdrawn through the first part 5 into the colon 8.

The piercing tip 43 of the cannula 40 is then engaged with and urged through the second part 6 of the colon wall 7 until the piercing tip 43 has penetrated through the colon wall 7 adjacent the second part 6. The second operating element 68 is then twisted relative to the first operating element 60 in the direction of the arrow F for urging the cam follower 76 through the first circumferential camming groove segment 82, for in turn aligning the cam follower 76 with the second longitudinally extending camming groove segment 79. The second operating element 68 is then urged relative to the first operating element 60 in the first direction, namely, in the direction of the arrow D, from the second state to the third state through the fourth predefined distance d₂ and the cam follower 76 travels along the second longitudinally extending camming groove segment 79 to the second stop element 86 thereof, for in turn urging the push rod 52 through the second predefined distance, see FIGS. 13a to 13c. At this stage, the second transverse member 22b has been urged from the suture accommodating chamber 47 through the distal end 42 of the cannula 40, and is located exteriorly of the colon 8 on the outer side 14 of the colon wall 7 adjacent the second part 6 with the suture 3 extending from the second transverse member 22b through the second part 6 of the colon wall 7 into the colon 8, see FIG. 13b.

The piercing tip 43 of the cannula 40 is then withdrawn through the second part 6 of the colon wall 7. The second operating element 68 is again twisted in the direction of the arrow F relative to the first operating element 60, for in turn urging the cam follower 76 through the second circumferential camming groove segment 83, in order to align the cam follower 76 with the third longitudinally extending camming groove segment 80.

The second operating element 68 is then urged in the first direction, namely, in the direction of the arrow D longitudinally relative to the first operating element 60 from the third state to the fourth state through the sixth predefined distance d₃ with the cam follower 76 travelling along the third longitudinally extending camming groove segment 80 to the third stop element 87 thereof, see FIGS. 14a to 14c. This in turn urges the distal portion 57 of the push rod 52, and in turn the hook 55 through the distal end 42 of the cannula 40 with the engagement hook 55 exposed in the colon 8, see FIG. 14b. With the first operating element 60 in the second state and the second operating element 68 in the fourth state, the loop 26 of the suture 3 is then engaged by the engagement hook 55, see FIG. 15b. The first and second operating elements 60 and 68 together are urged in the second direction, namely, in the direction of the arrow E from the second state of the first operating element 60 towards the first state of the first operating element 60, see FIGS. 16a to 16c. While the first and second operating elements 60 and 68 are being urged from the second state of the first operating element 60 in the direction of the arrow E, the loop 26 of the suture 3 is drawn into the cannula accommodating bore 41 of the tubular shield 35 until the adjustment element 25 of the suture 3 abuts the abutment face 38 of the tubular shield 35.

Further urging of the first and second operating elements 60 and 68 in the second direction, namely, in the direction of the arrow E result in the loop 26 of the suture 3 being urged through the adjustment element 25, which in turn results in the adjustment element 25 being urged in the direction of the arrow B along the loop 26, for in turn reducing the effective length of the suture 3 between the transverse members 22. This in turn results in the portions 5 and 6 of the colon wall 7 being drawn by the suture 3 towards each other, see FIG. 16b. The first and second operating elements 60 and 68 are urged in the second direction in the direction of the arrow E until the first and second parts 5 and 6 of the colon wall 7 are drawn tightly together to cause the polyp 12 to be everted and urged to the outer side 14 of the colon wall 7. If the movement of the first operating element 60 from the first state thereof to the second state thereof is insufficient for drawing the first and second parts 5 and 6 of the colon wall 7 together, the second operating element 68 is then urged in the second direction, namely, in the direction of the arrow E relative to the first operating element 60 from the fourth state thereof a sufficient distance through the third and second states to the first state if necessary with the cam follower 76 travelling along the camming groove 75, until the effective length of the suture 3 between the transverse members 22 is sufficiently small to urge the first and second parts 5 and 6 of the colon wall 7 together with the polyp 12 everted and urged to the external side of the colon wall 7.

At this stage the action of the abutment members 27 of the adjustment element 25 engaging the legs 20 of the loop 26 of the suture 3 with the predefined engagement force retains the adjustment element 25 securely engaged with the loop 26. Thereby, the suture 3 retains the parts 5 and 6 together with the polyp 12 everted and extending from the outer side 14 of the colon wall 7.

The first and second operating elements are then urged in the first direction, namely, in the direction of the arrow D for urging the cannula 40 distally through the distal end 37 of the tubular shield 35 and in turn for urging the engagement hook 55 through the distal end 42 of the cannula 40, so that the engagement hook 55 is exposed in the colon and can be disengaged from the loop 26 of the suture 3.

The suture 3 is retained engaged in the adjustment element 25 without slippage of the loop 26 through the adjustment element 25 in the direction of the arrow B due to the engagement force with which the abutment members 27 engage the legs 20 of the loop 26 of the suture 3.

The first and second operating elements 60 and 68 are again operated for returning the cannula 40 to the withdrawn state in the cannula accommodating bore 41 of the tubular shield 35, and for returning the push rod 52 to the withdrawn state in the cannula bore 46.

The tubular shield 35 of the suturing device 1 is then withdrawn from the colon 8 through the colonoscope (not shown), and the next suture 3 to be secured in the first and second parts 5 and 6 of the colon wall 7 is placed in the suture accommodating chamber 47 of the cannula 40 with the adjustment element 25 of the suture located in the adjustment element accommodating chamber 50 in the distal end of the tubular shield 35. The suturing device 1 is then ready to be entered into the colon 8 through the colonoscope for inserting the second suture 3 into the first and second parts 5 and 6 of the colon wall 7. The appropriate number of sutures 3 are inserted into the first and second parts 5 and 6 of the colon wall 7, for in turn everting the polyp 12 to the outer side 14 of the colon wall 7.

Subsequently, the polyp 12 is severed from the outer side 14 of the colon 8 using a surgical stapler during laparoscopic surgery through the abdominal wall.

Alternatively, it is envisaged that the suturing of the first and second parts 5 and 6 of the colon wall 7 for urging the polyp 12 internally into the colon could be carried out laparoscopically with the suturing device 1 by entering the tubular shield 35 of the suturing device 1 through a trocar into the abdominal cavity, and the suturing of the first and second parts 5 and 6 of the colon wall 7 would be carried out from the external side of the colon 8, instead of from the inside of the colon 8 as described above. In which case, it is envisaged that the polyp 12 would be everted into the colon and then would be severed using a surgical stapler deployed through a colonoscope.

Referring now to FIG. 21 there is illustrated a cannula also according to the invention indicated generally by the reference numeral 100 for releasably coupling to an actuator also according to the invention, only a portion of which is illustrated in FIG. 21, and is indicated generally by the reference numeral 101. The cannula 100 and the actuator 101 when coupled together form a suturing device (not shown) which is also according to the invention, and which is substantially similar to the suturing device 1. Accordingly, components of the cannula 100, and the portion of the actuator 101 which is illustrated, which are similar to components of the suturing device 1 are indicated by the same reference numerals.

The cannula 100 is coupled to the actuator 101 by a connecting element comprising a tubular connecting member 102 extending from a proximal end 104 of the cannula 100. The connecting member 102 is externally threaded for engaging an internally threaded socket 107 adjacent a distal end 109 of an elongated tubular connector 110 extending proximally from the distal end 109 to the first operating element of the actuator 101. The first operating element of the actuator 101 is similar to the first operating element of the actuator 34 of the suturing device 1, and the tubular connector 110 is secured to the first operating element for urging the cannula 100 between the withdrawn state within the tubular shield 35 and an extended state with the distal end 42 of the cannula 100 extending outwardly from the distal end 37 of the tubular shield 35.

A push rod 52 similar to the push rod 52 of the suturing device 1 extends from the second operating element of the actuator 101, which is similar to the second operating element of the actuator 34 of the suturing device 1. The push rod 52 extends through the tubular connector 110 and in turn through the socket 107, and into the cannula 100 through the connecting member 102. Accordingly, when the cannula 100 is secured to the tubular connector 110, and when the second operating element to which the push rod 52 is secured is in the first state with the push rod 52 in the withdrawn state, the engagement end 56 of the hook 55 defines with the distal portion of the bore 46 of the cannula 100 the suture accommodating chamber 47.

In use, the suture accommodating chamber 47 of the cannula 100 is charged with a suture 3 with the transverse members 22 of the suture 3 sequentially arranged longitudinally in the suture accommodating chamber 47. The cannula 100 is then secured to the tubular connector 110 with the first operating element 60 in the second state, and the second operating element in the first state. The first operating element 60 is then urged to the first state to withdraw the cannula 100 into the withdrawn state into the tubular shield 35 to define with the distal portion of the tubular shield 35, the adjustment element accommodating chamber 50 with the adjustment element 25 of the suture 3 located therein. With both the first and second operating elements 60 and 68 in the first state, the distal end 56 of the push rod 52 defines with the cannula, the suture accommodating chamber 47 with the transverse members 22 of the suture 3 located therein.

Thereafter, operation of the actuator 101 with the cannula 100 releasable coupled thereto is similar to operation of the suturing device 1.

Referring now to FIG. 22, there is illustrated a distal end indicated generally by the reference numeral 120 of an actuator according to another embodiment of the invention of a suturing device also according to the invention. The distal end 120 of the actuator according to this embodiment of the invention is substantially similar to the distal end of the actuator 101 of the embodiment described with reference to FIG. 21, and similar components are identified by the same reference numerals. The actuator according to this embodiment of the invention is substantially similar to the actuator 34 of the suturing device 1, and similar components are identified by the same reference numerals.

In this embodiment of the invention the push rod 52 does not terminate in a hook as in the case of the actuator 101 and the actuator 34 of the suturing device 1, but rather, in this embodiment of the invention the push rod 52 terminates in a plain engagement end 56.

The actuator 120 in this embodiment of the invention is configured for use separately with the cannula 100 of FIG. 21 and with a hook member indicated generally by the reference numeral 122. When the cannula 100 is releasably coupled to the tubular connector 110 by the connecting member 102 of the cannula 100 engaging the socket 107 of the tubular connector 110, the combined actuator 120 and the cannula 100 operates in a similar manner as described with reference to the cannula 100 and the actuator 101 of FIG. 21, with the exception that the engagement end 56 of the push rod 52 defines the suture accommodating chamber 47 in the distal portion 48 of the bore 46 of the cannula 100.

In this embodiment of the invention the actuator 120 with the cannula 100 is suitable only for inserting the two legs 20 of the suture 3 through the colon wall 7 of the parts 5 and 6 of the colon wall 7 with the transverse members 22 on the outer side 14 of the colon wall 7.

When all the sutures have been engaged in the colon wall 7, the tubular shield 35 along with the cannula 100 is withdrawn from the colon. The cannula 100 is then disengaged from the tubular connector 110, and the hook member 122 is then secured to the tubular connector 110. The hook member 122 comprises a tubular member 123. A hook 124 for engaging the loops 26 of the respective sutures 3 extends distally from a distal end 125 of the tubular member 123. A proximal end 126 of the tubular member 123 terminates in an externally threaded connector 127 for engaging the internally threaded socket 107 of the tubular connector 110 of the actuator 120. Accordingly, in this embodiment of the invention the hook member 122 being coupled to the tubular connector 110 is operated solely by the first operating element of the actuator 120.

In use, with the cannula 100 coupled to the tubular connector 110 of the actuator 120, and with the suture accommodating chamber 47 charged with a suture as already described with reference to the suturing device 1 and the actuator 101, the actuator 120 with the cannula connected thereto is ready for use. Use of the actuator 120 and the cannula 100 for inserting the sutures 3 into the parts 5 and 6 of the colon wall is similar to that described with reference to the suturing device of FIGS. 1 to 16 and the actuator of FIG. 21. Once the appropriate number of the sutures 3 have been inserted in the parts 5 and 6 of the colon wall 7, the cannula 100 is disengaged from the tubular connector 110, and the hook member 122 is coupled to the tubular connector 110 by engaging the externally threaded connector 127 in the internally threaded socket 107 of the tubular connector 110.

The tubular shield 35 with the hook member 122 withdrawn into the bore 41 of the tubular shield 35 by operating the first operating element 60 from the second state into the first state, is urged into the colon. Once the tubular shield 35 is in the colon 8, the first operating element is urged from the first state to the second state for urging the hook member 122 into the extended state with the hook 124 extending externally through the distal end 37 of the tubular shield 35. The loops 26 of the sutures 3 are individually engaged by the hook 124, and the hook member 122 is then urged from the extended state to the withdrawn state by urging the first operating element 60 from the second state to the first state for drawing the loop 26 of the suture 3 into the cannula accommodating bore 41 of the tubular shield 35, for in turn drawing the adjustment element 25 of the suture 3 against the abutment face 38 of the tubular member 35. Further urging of the operating element 60 towards the first state results in the loop 26 of the sutures 3 being urged in the direction of the arrow A through the adjustment element 25 for reducing the effective length of the sutures 3 between the transverse members 22, for in turn urging the parts 5 and 6 of the colon wall 7 together.

Alternatively, the loops 26 of more than one suture 3 may be engaged by the hook 124 of the hook member 122, and the loops 26 of those sutures 3 would be urged simultaneously into the cannula accommodating bore 41 of the tubular shield 35, for in turn simultaneously tightening the sutures 3.

Otherwise, the actuator 120 with the cannula 100 and with the cannula 100 replaced by the hook member 122 is similar to that described with reference to the suturing device 1.

In the embodiments of the invention described with reference to FIGS. 21 and 22 it is envisaged that the cannula 100 and the hook member 122 would be supplied separately of the actuators 101 and 120.

Referring now to FIGS. 23 to 26, there is illustrated a suture according to another embodiment of the invention indicated generally by the reference numeral 200. The suture 200 is substantially similar to the suture 3 described with reference to FIGS. 17 to 20, and similar components are identified by the same reference numerals. The suture 200 comprises an adjustment element 201. The only difference between the suture 200 and the suture 3 lies in the adjustment element 201, The main difference between the adjustment element 201 and the adjustment element 25 of the suture 3 lies substantially in the cross-sectional shape of the adjustment element 201. In this embodiment of the invention the transverse cross-sectional shape of the adjustment element 201 follows more closely the shape of a figure of eight or an hourglass than does the transverse cross-sectional shape of the adjustment element 25.

Otherwise, the suture 200 is similar to the suture 3 described with reference to FIGS. 17 to 20, and its use is likewise similar.

Referring now to FIGS. 27 to 30, there is illustrated a suture according to another embodiment of the invention indicated generally by the reference numeral 210. The suture 210 is substantially similar to the suture 3 described with reference to FIGS. 17 to 20, and similar components are identified by the same reference numerals. The suture 210 terminates at its respective opposite ends in respective anchor elements provided by transverse members 22, which are similar to the transverse members 22 of the suture 3. An adjustment element 211 is located on the loop 26 of the suture 210 and is urgeable along the loop 26 of the suture 210 for adjusting the effective length of the suture 210 between the transverse members 22 thereof. The only difference between the suture 210 and the suture 3 lies in the adjustment element 211.

In this embodiment of the invention the retainer of the adjustment element 211 comprises a pair of spaced apart abutment members 212 which terminate in corresponding spaced apart abutment edges 214 which in turn define therebetween a suture accommodating gap 215 for accommodating the legs 20 of the loop 26 of the suture 3 therethrough. The abutment edges 214 of the abutment members 212 are configured to permit movement of the adjustment element 211 along the legs 20 of the loop 26 of the suture 211 in the direction of the arrow B, and to grip the legs 20 of the loop 26, and thus resist and prevent movement of the adjustment element 211 along the legs 20 of the loop 26 in the direction of the arrow A. Accordingly, when the adjustment element 211 is urged along the legs 20 of the loop 26 in the direction of the arrow B to the selected location with the effective length of the suture 3 between the transverse members 22 at the desired effective length, the abutment edges 214 engage and grip the suture 211 for preventing movement of the adjustment element 211 in the direction of the arrow A along the loop 26.

The abutment members 212 are joined by a first cross-member 217 formed by an arcuate plate similar to the arcuate plate of the first cross-member 16 of the suture 3. A suture accommodating opening 218 extends through the cross-member 217, and accommodates the legs 20 of the loop 26 of the suture 210 as it exits the suture accommodating gap 215. The first cross-member 217 is of arcuate shape when viewed through the suture accommodating gap 215, and is of a spring type material, in this case stainless steel material, which is configured to bias the abutment edges 214 towards each other for engaging and gripping the legs 20 of the loop 26 of the suture 210 with a predefined engagement force sufficient to overcome the pulling force induced in the legs 20 of the suture 3 between the abutment edges 214 of the abutment members 212 and the transverse members 22 by the parts 5 and 6 of the colon wall 7 acting on the transverse members 22 of the suture 3 when the parts 5 and 6 are secured tightly together by the suture 3.

Additionally, the abutment edges 214 of the abutment members 212 are returned into an area 219 defined by and substantially circumscribed by the arcuate cross-member 217. In this embodiment of the invention the adjustment element 211 is of transverse cross-section of shape substantially similar to a half of a figure of eight or a half of an hourglass shape.

Otherwise, the suture 210 is similar to the suture 3 described with reference to FIGS. 17 to 20, and its use is likewise similar.

Referring now to FIGS. 31 to 35, there is illustrated a suture according to another embodiment of the invention indicated generally by the reference numeral 220. The suture 220 is substantial similar to the suture 3 described with reference to FIGS. 17 to 20, and similar components are identified by the same reference numerals. The only difference between the suture 220 and the suture 3 lies in an adjustment element 221 of the suture 220.

In this embodiment of the invention the adjustment element 221 comprises a plate member 223 of stainless steel. A retainer, which in this embodiment of the invention comprises a pair of retaining members 224 formed in the plate member 223 engage the legs 20 of the loop 26 of the suture 220 extending through the adjustment element 221, as will be described below. The retaining members 224 are formed in and from the plate member 224 by partially severing the retaining members 224 from the plate member 223 along opposite sides 227 of the retaining members 224. The retaining members 224 extend from the plate member 223 adjacent proximal ends 225 thereof. The retaining members 224 extend from their proximal ends 225 and are severed from each other along their respective distal ends 226. Recesses 228 are formed in the distal ends 226 of the respective retaining members 224. The recesses 228 of the retaining members 224 defines respective abutment edges 229 which define therebetween a suture accommodating gap 230 for accommodating the legs 20 of the loop 26 of the suture 3 therethrough.

The retaining members 224 are slightly angled outwardly of the plane of the plate member 223 against the natural inherent springiness of the plate member 223, and are spring biased by the natural springiness of the plate member 223 from the outwardly angled state to a state lying substantially in the plane of the plate member 223. Thus, the retaining members 224 are configured to spring urge the abutment edges 229 into engagement with the legs 20 of the loop 26 of the suture 220, and the abutment edges 229 are configured to permit slideable movement of the adjustment element 221 along the legs 20 of the loop 26 of the suture 220 in the direction of the arrow B, and to grip the legs 20 of the loop 26, and thereby to resist movement of the adjustment element 221 along the loop 26 in the direction of the arrow A. Accordingly, when the adjustment element 221 has been urged along the loop 26 in the direction of the arrow B to a desired selected location along the loop 26 with the effective length of the suture 220 between the transverse members 22 of the desired effective length for retaining the parts 5 and 6 of the colon wall 7 together, the gripping action of the abutment edges 229 on the legs 20 of the loop 26 is such to prevent any movement of the adjustment element 221 along the loop 26 in the direction of the arrow A.

In this embodiment of the invention by providing the recesses 228 in the distal ends 226 of the retaining members 224, the legs 20 of the loop 26 of the suture 220 are retained in the suture accommodating gap 22 without any danger of the legs 20 becoming impaled on the retaining members 224 between the retaining members 224 and the plate member 223 along the side edges 227 of the retaining members 224.

Otherwise, the suture 220 is similar to the suture 3 of FIGS. 17 to 20, and its use is also similar to that of the suture 3.

It is envisaged that the suturing devices according to the invention which have been described with reference to FIGS. 1 to 16, 21 and 22, and in particular, though not limited to the suturing device 1 described with reference to FIGS. 1 to 16, may be provided without the engagement element, namely, the engagement hook 55. In which case, the urging element for urging the transverse members 22 of the suture 3, or any of the other sutures described with reference to FIGS. 23 to 35 from the suture accommodating chamber 47 would be provided by any suitable wire type push rod, which would terminate in a plain engagement end, similar to the engagement end 56, in which the push rod 52 terminates in the actuator 120 of the embodiment described with reference to FIG. 22. The plain engagement end 56 of the push rod 52 would engage the proximal most one of the transverse members 22 of the suture 3 in the suture accommodating chamber 47 of the cannula 40, and the transverse members 22 would be sequentially urged from the suture accommodating chamber 47 by the push rod 52. In such cases where the suturing devices according to the invention are provided without the engagement hook 55 or other suitable engagement element, a separate suture securing instrument would be provided for drawing the loops 26 through the respective adjustment elements of the sutures 3 or for drawing the loops 26 of the sutures through the adjustment elements of the other sutures described with reference to FIGS. 23 to 35. Additionally, it is envisaged that in cases where the engagement hook or other engagement element is omitted from the urging element, the urging element may be configured to be urgeable through the first and second predefined distances only, and in turn, the second operating element would be configured to operate through the third and fourth predefined distances only by configuring the second operating element to be operable from the first state thereof to the second state thereof through the third predefined distance, and from the second state to the third state through the fourth predefined distance.

Referring now to FIGS. 36 and 37 there is illustrated a suture securing instrument indicated generally by the reference numeral 250, which in some embodiments of the suturing device also forms a part of the invention along with the suturing device. The suture securing instrument 250 comprises a handle 252 and a tubular member 253 extending from the handle 252 adjacent a distal end 265 thereof. In this embodiment of the invention the tubular member 253 comprises a flexible Bowden cable having a bore 254 extending therethrough for accommodating a carrier, in this embodiment of the invention an elongated carrier cable 255 extending therethrough.

The carrier cable 255 terminates at a distal end 258 in an engagement element, in this embodiment of the invention an engagement hook 259, which is configured for engaging the loops 26 of the sutures 3, and also the sutures described with reference to FIGS. 23 to 35. The carrier cable 255 extends through and is slideable longitudinally in the bore 254 of the tubular member 253. The carrier cable 255 terminates in a proximal end (not shown) in an elongated longitudinally extending bore (also not shown) formed within the handle 252.

An actuator member (not shown) terminating at its respective opposite ends in a pair of finger-grip elements 260 extends transversely through the handle 252 through respective longitudinally extending elongated guide slots 262 on respective opposite sides of the handle 252. The actuator member (not shown) is slideable longitudinally in the direction of the arrows X and Y along the handle 252 in the guide slots 262. The actuator member (not shown) extends across the elongated bore (not shown) extending longitudinally through the handle 252, and is secured to the proximal end (not shown) of the carrier cable 255 for slideably urging the carrier cable 255 longitudinally through the bore 254 in the directions of the arrows X and Y, as the finger-grip elements 260 of the actuator member (not shown) are urged longitudinally along the handle 252 in the directions of the arrows X and Y.

The elongated longitudinally extending bore (not shown) which extends through the handle 252 communicates with the bore 254 extending through the tubular member 253, and the proximal end (not shown) of the carrier cable 255 extends into the elongated bore (not shown) extending longitudinally through the handle 252. The finger-grip elements 260 are urgeable along the handle 252 in the direction of the arrow X from a first state illustrated in FIG. 36 with the finger-grip elements 260 and the actuator (not shown) located adjacent a proximal end 264 of the handle 252 with the engagement hook 259 in a withdrawn state withdrawn into and located within the bore 254 of the tubular member 253, to a second state adjacent the distal end 265 of the handle 252 with the engagement hook 259 in an extended state extending outwardly of a distal end 266 of the tubular member 253 for engaging the loop 26 of, for example, the suture 3.

The distal end 266 of the tubular member 253 terminates in an abutment face 267 similar to the abutment face 38 in which the tubular shield 35 of the suturing device 1 terminates. The abutment face 267 of the tubular member 253 fulfils a similar function as that of the abutment face 38 of the tubular shield 35 of the suturing device 1. Accordingly, as the finger-grip elements 260 are urged in the direction of the arrow Y from the second state to the first state for withdrawing the engagement hook 259 from the extended state to the withdrawn state into the bore 254 of the tubular member 253, the loop 26 of, for example, the suture 3 engaged by the engagement hook 259 is withdrawn into the tubular member 253. As the loop 26 of the suture 3 is being withdrawn into the tubular member 253, the adjustment element of the suture 3 abuts the abutment face 267 of the tubular member 253, and further urging of the finger grip elements 260 in the direction of the arrow Y results in the loop 26 of the suture 3 being drawn in the direction of the arrow A through the adjustment element 25, and in turn the adjustment element 25 being urged along the legs 20 of the loop 26 in the direction of the arrow B towards the transverse members 22 of the suture 3, for in turn pulling the parts 5 and 6 of the colon wall 7 adjacent each other for everting the polyp to the outer side wall 14 of the colon wall 7.

In use, when the appropriate number of sutures 3 have been inserted into the colon wall 7 with the legs 20 of the respective sutures inserted through the parts 5 and 6 of the colon wall 7 and with the transverse members 22 on the outer side 14 of the colon wall 7, the tubular member 253 of the suture securing instrument 250 is entered rectally into the colon 8 with the finger-grip elements 260 in the first state, and with the engagement hook 259 in the withdrawn state. The suture securing instrument 250 is then manoeuvred in the colon until the distal end 266 of the tubular member 253 is located adjacent the loop 26 of the suture 3 to be engaged by the engagement hook 259. The finger-grip elements 260 are then urged from the first state to the second state for urging the engagement hook 259 from the withdrawn state to the extended state with the engagement hook 259 extending distally from the distal end 266 of the tubular member 253.

The engagement hook 259 is then engaged in the loop 26 of the suture 3. The finger-grip elements 260 are then urged in the direction of the arrow Y from the second state towards the first state for drawing the engagement hook 259 from the extended state into the tubular member 253 for in turn drawing the loop 26 of the suture 3 into the tubular member 253. As the engagement hook 259 is withdrawing the loop 26 of the suture 3 into the tubular member 253, the adjustment element 25 of the suture 3 engages the abutment face 267 defined by the distal end 266 of the tubular member 253. Once the adjustment element 25 has engaged the abutment face 267, further urging of the loop 26 into the tubular member 253 results in the adjustment element 25 being urged in the direction of the arrow B along the loop 26 of the suture 3 towards the transverse members 22 of the suture 3. This as described with reference to the suturing device 1 results in the parts 5 and 6 of the colon wall 7 being drawn together for everting the polyp 12 to the outer side 14 of the colon wall 7. The finger-grip elements 260 are urged in the direction of the arrow Y towards the first state thereof until the parts 5 and 6 of the colon wall 7 have been adequately drawn together.

Thereafter, the hand-grip elements 260 are urged in the direction of the arrow X to the second state for urging the engagement hook 259 to the extended state extending distally from the distal end 266 of the tubular member 253. The engagement hook 259 is then disengaged from the loop 26 of the just secured suture 3. The engagement hook 259 is then engaged with the loop 26 of the next suture 3 to be secured, and operation of the suture securing instrument 250 continues as already described until all the sutures have been secured by the suture securing instrument 250.

The tubular member 253 of the suture securing instrument 250 is then withdrawn from the colon of the subject.

Should it be desired to secure a plurality of the sutures 3 simultaneously, the loops 26 of the sutures 3 are sequentially engaged by the engagement hook 259. When all the loops 26 have been engaged by the engagement hook 259, the finger grip elements are urged from the second state in the direction of the arrow Y for drawing the loops 26 into the tubular member 253, for in turn urging the adjustment elements 25 of the sutures along the legs 20 of the loops 26 in the direction of the arrow B, for in turn drawing the parts 5 and 6 of the colon 8 together.

While the suture securing instrument 250 has been described for use with the suture 3, it will be appreciated that the suture securing instrument may also be used with the sutures described with reference to FIGS. 23 to 35 or with any other suitable suture.

While a separate suture securing instrument has been described for securing the sutures which is separate to the suturing device according to the invention, it will be readily apparent to those skilled in the art that any other suitable suture securing instrument besides the suture securing instrument 250 may be provided for securing the sutures after they have been inserted by the securing device.

While the first and second operating elements of the suturing device have been described as being moveable in first and second directions between the first and second states, in the case of the first operating element and between first, second, third and fourth states in the case of the second operating element, it will be readily understood by those skilled in the art that the first and second operating elements may be urged in different directions besides the first and second direction, and it is envisaged that in some embodiments of the invention that the second operating element may be urged in different directions as the second operating element is being urged from the first to the fourth states. For example, the second operating element may be operated from the first state to the second state in one direction, and could then be operated from the second state to the third state in another different direction, which would be different to the first direction, for example, it may be angled at an angle greater than zero relative to the first direction. The second operating element could then be operable from the third state to the fourth state in a still further different direction, which could also be angled at an angle greater than zero relative to the direction of movement of the second operating element from the second state to the first state, and could also be angled at an angle greater than zero relative to the direction of movement of the second operating element from the first state to the second state.

Instead of the second operating element being urgeable in a longitudinal direction, for example, relative to the handle, relative to the first operating element, or relative to the urging element, as the second operating element is being urged through the third, fourth and sixth predefined distances from the first state thereof to the second state thereof, and in turn from the second state thereof to the third state thereof, and then from the third state thereof to the fourth state thereof, it is envisaged that the second operating element may be rotatably mounted, and would be rotatable from the first state to the second state for in turn urging the urging element through the first predefined distance, and the second operating element would then be rotatable from the second state thereof to the third state thereof for urging the urging element through the second predefined distance, and further, the second operating element would be rotatable from the third state thereof to the fourth state thereof for urging the urging element through the fifth predefined distance.

It is also envisaged that the second operating element may be rotatable in the same direction as it is being urged through the first, second, third and fourth states, or alternatively, it is envisaged that the second operating element may be rotatable in, for example, a clockwise direction as it is being urged from the first state thereof to the second state thereof, and in a counter-clockwise direction as it is being urged from the second state thereof to the third state thereof, and again in a clockwise direction as it is being urged from the third state thereof to the fourth state thereof, or vice versa, whereby initially, the second operating element would be urgeable from the first state to the second state in a counter-clockwise direction, and so on. It is also envisaged that a stop means would be provided at the end of each rotational traverse between the respective first and second states, the second and third states and the third and fourth states. It is also envisaged that the second operating element may be urgeable through a short distance, for example, a longitudinal distance between each rotational operation of the second operating element when its rotational movement terminates when it has reached the second state and the third state, before the second operating element can be urged from the relevant second state to the third state and from the relevant third state to the fourth state.

Needless to say, instead of the second operating element being operable between the first, second, third and fourth states, in a rotational direction, the second operating element could be rotatable in a helical manner between the first, second, third and fourth states thereof.

Needless to say, the first operating element may also be rotatable between the first state thereof and the second state thereof, and may be rotatable helically between the first and second states.

It is also envisaged that the path of movement or paths of movement of the second operating element from the fourth state back to the first state may be different to the paths of movement of the second operating element from the first state to the fourth state.

Similarly, it is envisaged that the direction of movement of the first operating element from the first state to the second state thereof could also be different to any of the directions of movement of the second operating element from the first state to the fourth state. It is also envisaged that the path of movement of the first element from the second to the first state may be different to the path of movement of the first operating element from the first state to the second state thereof. It is envisaged that a quick return path may be provided for returning the first operating element from the second state to the first state, and similarly, it is envisaged that a quick return path may be provided for returning the second operating element from the fourth or the third state, as the case may be to the first state thereof.

It will also be appreciated that while it is desirable it is not essential that the second operating element be urged in different directions between the movement of the second operating element from the first to the second state, and from the second to the third state, and similarly, it is envisaged that it may not be necessary that the second operating element be urged in a different direction between the urging of the second operating element from the second state to the third state and from the third state to the fourth state. Further, while it is desirable, it is not essential that the movement of the second operating element in the different directions between the movement of the second operating element from the first to the second state and the second to the third state, and from the second state to the third state and from the third state to the fourth state being in a circumferential direction, the different direction may be in any direction apart from a circumferential direction.

It is also envisaged that the length of the third predefined distance traversed by the second operating element as it is being urged from the first state thereof to the second state thereof may be the same or different to the length of the first predefined distance through which the urging element is being urged, and whether or not the lengths of the third and first predefined distances are the same or different, will depend on the type of movement of the second operating element as it is being moved from the first state thereof to the second state thereof. For example, in a case where the second operating element is being urged with rectilinear motion longitudinally relative to the handle or the first operating element or indeed, relative to the urging element or the cannula, then, the third and first predefined distances would most likely be of the same length. However, if the second operating element were being urged from the first state thereof to the second state thereof with rectilinear or non-rectilinear motion at an angle to the direction of movement of the urging element, for example, if the second operating element were being rotated with, for example, helical motion, then the length of the third predefined distance travelled by the first operating element between the first and second states thereof most likely would be different to the length of the distance travelled by the urging element over the first predefined distance. Similarly, the fourth predefined distance travelled by the second operating element between the second state thereof and the third state thereof, may likewise be the same as or different to the length of the second predefined distance, and similarly, the length of the sixth predefined distance travelled by the second operating element between the third state thereof and the fourth state thereof may also be the same as or different to the length of the fifth predefined distance travelled by the urging element, depending on the type of movement of the second operating element between the second and the third states thereof and between the third and the fourth states thereof.

Additionally, it is envisaged that the distance travelled by the first operating element between the first state and the second state may be different to the distance travelled by the cannula as it is being urged by the first operating element between the withdrawn state and the extended state, and whether or not the two distances are the same or different, would depend on the type of motion and the direction thereof of the first operating element between the first state thereof and the second state thereof.

While the urging element has been described as comprising a push rod, while this is desirable, it is not essential, and other suitable urging elements may be provided. It will be readily apparent to those skilled in the art that while it is desirable it is not essential that the engagement element for engaging the loop of the suture be secured to the distal end of the urging element. It will be appreciated that the engagement element may be operated independently of the urging element, and furthermore, it is envisaged that the engagement element may be operated independently of the cannula.

It is also envisaged that while the sutures have been described as comprising a single suture extending between a pair of anchor elements, in some embodiments of the invention it is envisaged that the suture may be severed intermediate its ends, in which case one part of the suture extending from one of the anchor elements would be non-adjustably connected to the adjustment element and the other part of the suture extending from the other one of the anchor elements would extend through the suture accommodating gap of the adjustment element, and the adjustment element would be moveable along that part of the suture extending through the suture accommodating gap for adjusting the effective length of the suture between the two anchor elements.

It is also envisaged that each suture may be provided with a plurality of nodules spaced apart along the length of the suture which would be sequentially engageable with the retainer of the adjusting element to prevent movement of the adjustment element along the loop in a direction away from the anchor elements. The retainer would be configured to permit movement of the adjustment element along the or each leg of the loop in a direction towards the anchor elements, and to prevent movement along the or each leg of the loop in a direction away from the anchor elements.

Needless to say, any other suitable operating elements besides the first and second operating elements may be provided. It will of course be appreciated that the operating elements need not necessarily be mounted coaxially with the carrier, for example, the first and second operating elements could be formed by slider elements, such as, for example, slider members, which would be slideably mounted in the carrier and would extend therefrom.

It will be appreciated that where the distal cannula portion is provided separately from the actuator, that the distal cannula portion may be releasably attachable to the actuator by any suitable means besides screw threads, for example, the distal cannula portion may be releasably securable to the actuator by a bayonet fitting, or by any suitable press fit or the like.

It is also envisaged in the adjustment element of the suture, that instead of both of the abutment members or retaining members being spring biased to engage the leg or legs of the loop of the suture, in some cases only one of the abutment members or the retaining members may be spring biased for urging the leg or legs of the loop against the other one of the abutment members or retaining members, for in turn engaging the leg or legs of the loop between the two abutment members or the two retaining members.

While the suturing device has been described for everting a polyp in a colon, it will be readily apparent to those skilled in the art that the suturing device may be used for everting any other element in a vessel, lumen or cavity for urging that element from one side of the vessel, lumen or cavity to the other side thereof. It will also be appreciated that the suturing device according to the invention may be used as well for everting small cancers to cause the small cancer to be urged from one side of a colon, lumen, vessel or cavity to the other side, for example, from the inside to the outside of the colon, lumen, vessel or cavity, or vice versa. Needless to say, it will be appreciated that the suturing device may be used for suturing any two parts of a human or animal body together, and in particular, for suturing any two parts of a lumen, vessel or cavity, for example, adjacent sides of an incision in tissue of a lumen, vessel or cavity, or an incision in tissue in any part of a human or animal body. Further, it will be appreciated that the suturing device may be used for suturing in any lumen, vessel or cavity in the human or animal body, and the tubular shield of the suturing device may be entered into that lumen, vessel or cavity through any suitable orifice of the human or animal body, for example, the tubular shield of the suturing device may be entered orally, nasally or rectally into the human or animal body, and in other embodiments of the invention, the suturing device may be entered into the lumen, vessel or cavity directly through an incision made in the human or animal body, or through a trocar or any other suitable instrument through which access to the lumen, vessel or cavity may be gained.

It is also envisaged that as well as the distal cannula portion being releasably securable to the actuator, it is envisaged that the second carrier element, namely, the tubular shield or a part thereof may be releasably securable to the actuator. The releasable securing of the tubular element or the portion thereof to the actuator could be achieved by any suitable releasable connecting means, for example, by screw threads, by a bayonet type connection, by a press fit or the like.

It is also envisaged that the suture accommodating gap defined by the retainer or the abutment members of the adjustment element may be defined as a suture accommodating bore extending through the retainer or defined by the abutment members or extending through the adjustment element, and the suture accommodating bore would typically comprise a tapered bore extending between a first end and a second end, the diameter of the suture accommodating bore adjacent the first end would be greater than the diameter of the suture accommodating bore adjacent the second end, and preferably, the material defining the suture accommodating bore adjacent at least the second end thereof would comprise a resilient material, which would be configured to contract into the suture accommodating bore adjacent the second end thereof. In cases where the suture comprises a plurality of spaced apart nodules located spaced apart along the suture, the transverse cross-section of the nodules would be greater than the transverse cross-section of the suture accommodating bore adjacent the second end thereof, and advantageously, the resilience of the material defining the second end of the suture accommodating bore would be such as to permit movement of the nodules through the second end of the suture accommodating bore in a direction from the first end to the second end thereof, and to prevent movement of the nodules through the second end of the suture accommodating bore in the direction from the second end of the suture accommodating bore to the first end thereof. It is also envisaged that the resilience of the material defining the second end of the suture accommodating bore would be such as to engage the nodules with a snap-fit type action.

It is also envisaged that in cases where the suture comprises spaced apart nodules, each nodule would comprise a bead, and preferably, a spherical bead, and each bead would be of diameter greater than the diameter of the suture accommodating bore adjacent the second end thereof where the suture accommodating bore is of its smallest diameter adjacent the second end thereof. It is also envisaged that the spaced apart heads forming the nodules on the legs of the sutures may be of conical or pyramid shape, or frustoconical or frustopyramidal shape with the larger diameter ends thereof facing the corresponding anchor element.

It is also envisaged that the nodules on the suture may be of a dissolvable material, which would be dissolvable in a human or animal body.

While the components of the suturing devices described with reference to FIGS. 1 to 16, 21 and 22, the sutures described with reference to FIGS. 17 to 20 and 23 to 35, and the suture securing instrument described with reference to FIGS. 36 and 37 have been described as being of specific materials, it will be readily apparent to those skilled in the art that the components of the suturing devices, the sutures and the suture securing instrument may be of any suitable materials. Indeed, in some embodiments of the invention instead of stainless steel materials, other suitable metal materials and metal alloys may be used, and in other embodiments of the invention instead of stainless steel materials, plastics and polymer materials may also be used. Needless to say, handles, operating elements, actuator elements and the like of the suturing devices and the suture securing instrument typically, may be of a plastics or polymer material, although, it will be readily apparent to those skilled in the art that these components may be of metal material and metal alloy materials.

While the sutures described according to the invention have been described as comprising a medical grade nylon material, any other suitable materials, plastics or polymer materials, or other natural occurring materials may be provided. However, in general, such materials for the suture should be of a medical grade material, and in some embodiments of the invention dissolvable materials, and in others, non-dissolvable materials.

1-183. (canceled)

184. A suturing device for applying a suture, the suture terminating at its opposite ends in respective anchor elements, the suturing device comprising:
a cannula having a bore extending longitudinally therethrough for accommodating the suture therein and terminating adjacent a distal end thereof in a piercing point,
an urging element urgeable into the cannula bore for urging the anchor elements of the suture sequentially from the cannula bore through the distal end thereof,
a first operating element coupled to the cannula, and
a second operating element coupled to the urging element,
the second operating element being operable for urging the urging element in the cannula bore in a distal direction to sequentially urge the anchor elements of the suture from the cannula.

185. A suturing device as claimed in claim 184 in which an engagement element is provided for engaging a portion of the suture, and one of the first and second operating elements is operably coupled to the engagement element, and preferably, the engagement element is non-releasably coupled to the one of the first and second operating elements, and advantageously, the engagement element is releasably coupled to the second operating element, and preferably, an abutment element is provided for abutting an adjustment element of the suture, and one of the first and second operating elements is operable for urging the engagement element in a proximal direction to withdraw a portion of the suture engaged by the engagement element through the adjustment element of the suture with the adjustment element abutting the abutment element to shorten the effective length of the suture between the anchor elements, and advantageously, the cannula is non-releasably coupled to the first operating element, or alternatively, the cannula is releasably coupled to the first operating element, and preferably, the urging element is urgeable in a distal direction relative to the cannula for urging the anchor elements of the suture from the cannula, and advantageously, the urging element is urgeable from a withdrawn state within the cannula bore in the distal direction relative to the cannula for urging the anchor elements of the suture from the cannula, and preferably, in the withdrawn state of the urging element, a distal end thereof defines with the cannula a suture accommodating chamber for accommodating the respective anchor elements of the suture, and advantageously, the suture accommodating chamber is defined in the cannula bore between the distal end of the cannula and the distal end of the urging element, and preferably,
the suture accommodating chamber is configured to accommodate the anchor elements of the suture sequentially along the suture accommodating chamber, and advantageously, the length of the suture accommodating chamber is substantially equal to the sum of the lengths of the respective anchor elements, and preferably, a suture accommodating slot extends longitudinally into the cannula from the distal end thereof for accommodating a portion of the suture therethrough from the suture accommodating chamber, and advantageously, the urging element is urgeable from the withdrawn state distally through a first predefined distance for urging a first one of the anchor elements of the suture from the cannula, and preferably, the length of the first predefined distance is substantially equal to the length of the first one of the anchor elements of the suture to be urged from the cannula, and advantageously, the urging element is urgeable distally through a second predefined distance for urging a second one of the anchor elements of the suture from the cannula, and preferably, the length of the second predefined distance is substantially equal to the length of the second one of the anchor elements of the suture to be urged from the cannula, and advantageously, the urging element is urgeable sequentially through the first and second predefined distances, and preferably, the second operating element is operable from a first state thereof to a second state thereof for urging the urging element through the first predefined distance, and advantageously, the second operating element is moveable from the first state thereof to the second state thereof, and a first stop element is provided for terminating movement of the second operating element from the first state thereof to the second state thereof when the second operating element has reached the second state, and preferably, the second operating element is operable from the second state thereof to a third state thereof for urging the urging element through the second predefined distance.

186. A suturing device as claimed in claim 184 in which the second operating element is moveable from the second state thereof to the third state thereof, and a second stop element is provided for terminating movement of the second operating element from the second state thereof to the third state thereof when the second operating element has reached the third state thereof, and preferably,
the second operating element is moveable from the first state thereof to the second state thereof through a third predefined distance for urging the urging element distally through the first predefined distance, and advantageously, the second operating element is moveable from the first state thereof to the second state thereof with rectilinear motion, and preferably, the second operating element is moveable from the first state thereof to the second state thereof relative to the first operating element, and advantageously,
the second operating element is urgeable from the first state thereof to the second state thereof in a first direction, and preferably, the second operating element is operable from the second state thereof to the third state thereof through a fourth predefined distance for urging the urging element distally through the second predefined distance, and advantageously, the second operating element is moveable from the second state thereof to the third state thereof, and preferably, the second operating element is moveable from the second state thereof to the third state thereof with rectilinear motion, and advantageously,
the second operating element is moveable from the second state thereof to the third state thereof relative to the first operating element, and preferably, the second operating element is moveable from the second state thereof to the third state thereof in the first direction, and advantageously, the distal end of the cannula defines the abutment element for abutting the adjustment element of the suture, and preferably,
a carrier is provided for carrying the first and second operating elements, and advantageously, the carrier comprises a first carrier element and a second carrier element extending distally from the first carrier element, and preferably, the first carrier element is configured to carry the first and second operating elements, and advantageously, the first carrier element forms a handle, and preferably, the second carrier element terminates adjacent a distal end thereof in the abutment element for abutting the adjustment element of the suture, and advantageously, the second carrier element is configured to slideably accommodate the cannula therein, and the cannula is urgeable from a withdrawn state within the second carrier element to an extended state extending distally outwardly from the distal end of the second carrier element, and preferably, the second carrier element defines with the distal end of the cannula in the withdrawn state an adjustment element accommodating chamber for accommodating the adjustment element of the suture, and advantageously, the cannula is configured to urge the adjustment element of the suture from the adjustment element accommodating chamber through the distal end of the second carrier element as the cannula is urged from the withdrawn state to the extended state, and preferably,
the first operating element is urgeable from a first state thereof to a second state thereof for urging the cannula from the withdrawn state within the second carrier element to the extended state, and advantageously, the second operating element is urgeable with the first operating element when the first operating element is being urged from the first state thereof to the second state thereof for urging the cannula from the withdrawn state within the second carrier element to the extended state, and preferably,
the first operating element is urgeable from the second state thereof to the first state thereof for urging the cannula from the extended state to the withdrawn state within the second carrier element, and advantageously, the second operating element is urgeable with the first operating element when the first operating element is being urged from the second state thereof to the first state thereof for urging the cannula from the extended state to the withdrawn state within the second carrier element, and preferably,
the second operating element is urgeable from the third state thereof through the second state thereof to the first state thereof relative to the first operating element for urging the urging element in a proximal direction to the withdrawn state thereof, and advantageously, the second operating element is urgeable from the second state thereof to the first state thereof and from the third state thereof to the second state thereof in a second direction, and the first operating element is urgeable from the second state thereof to the first state thereof in the second direction, the second direction being opposite to the first direction, and preferably, the second carrier element comprises an elongated tubular shield, and advantageously,
the elongated tubular shield comprises a flexible tubular shield, and preferably, the engagement element is coupled to the urging element adjacent the distal end thereof.

187. A suturing device as claimed in claim 184 in which the urging element is urgeable through the cannula to an extended state with the engagement element located distally from the distal end of the cannula for engaging the portion of the suture, and preferably, the urging element is urgeable distally in the cannula to the extended state by the second operating element, and advantageously,
the urging element is urgeable distally through a fifth predefined distance to the extended state after the urging element has been urged through the second predefined distance.

188. A suturing device as claimed in claim 184 in which the second operating element is moveable from the third state thereof to a fourth state thereof for urging the urging element through the fifth predefined distance, and a third stop element is provided for terminating movement of the second operating element from the third state thereof to the fourth state thereof when the second operating element has reached the fourth state thereof, and preferably, the second operating element is moveable from the third state thereof to the fourth state thereof through a sixth predefined distance for urging the urging element distally through the fifth predefined distance, and advantageously, the second operating element is urgeable from the third state thereof to the fourth state thereof relative to the first operating element, and preferably, the second operating element is urgeable in the second direction for urging the urging element in a proximal direction from the extended state for urging the engagement element in a proximal direction, for in turn urging the portion of the suture engaged by the engagement element through the adjustment element, and advantageously, the first operating element is urgeable in the second direction with the second operating element as the second operating element is being urged in the second direction for urging the urging element from the extended state, and preferably, the second operating element is moveable relative to the first operating element for urging the urging element in a proximal direction to the withdrawn state, and advantageously, the urging element is urgeable from the extended state for in turn urging the engagement element proximally for urging the adjustment element of the suture into abutting engagement with the abutment element as the portion of the suture is urged proximally through the adjustment element by the engagement element, and preferably, the engagement element is coupled to the first operating element, and the first operating element is urgeable from the second state thereof to the first state thereof for urging the engagement element in a proximal direction for drawing the portion of the suture through the adjustment element, and advantageously, the engagement element is releasably coupleable to the one of the first operating element and the second operating element, and preferably, the engagement element comprises a hook for engaging a loop of the suture for urging the loop of the suture through the adjustment element, and advantageously, the first operating element is slideably mounted on the first carrier element.

189. A suturing device as claimed in claim 184 in which a guide means is provided for guiding movement of the second operating element relative to the first operating element between the first and second states of the second operating element, between the second and third states, and between the third and fourth states of the second operating element, and preferably, the guide means is adapted to guide movement of the second operating element relative to the first operating element through the third and fourth predefined distances, and advantageously, the guide means is adapted to guide movement of the second operating element relative to the first operating element through the sixth predefined distance.

190. A suturing device as claimed in claim 189 in which the guide means is configured to guide the second operating element to move relative to the first operating element in a third direction different to the first and second direction between the travel of the second operating element relative to the first operating element through the third predefined distance and the fourth predefined distance, and preferably, the guide means is configured to guide the second operating element relative to the first operating element in the third direction or in a fourth direction different to the first and second direction between the travel of the second operating element relative to the first operating element through the fourth predefined distance and the sixth predefined distance, and advantageously, the third direction extends transversely of the first direction, and preferably, the fourth direction extends transversely of the first direction, and advantageously, the fourth direction is in the same direction as the third direction.

191. A suturing device as claimed in claim 189 in which the guide means comprises a camming groove located on one of the first and second operating elements, and a cam follower located on the other one of the first and second operating elements, and preferably, the first operating element is keyed relative to the first carrier element for preventing rotation of the first operating element relative to the first carrier element, and advantageously, the first operating element is coaxial with the first carrier element, and preferably, the second operating element is coaxial with the first operating element, and advantageously, the urging element comprises an elongated push rod extending from the second operating element, and preferably, the push rod comprises a flexible material, and advantageously, the cannula is coupled to the first operating element by an elongated tubular connecting member, and preferably, the urging element extends through the tubular connecting member, and advantageously, the cannula comprises an elongated flexible member, and preferably, the cannula is releasably coupleable to the tubular connecting member, and advantageously, the engagement element is coupled to the tubular connecting member, and preferably, the engagement element is releasably coupleable to the tubular connecting member.

192. A suture terminating at respective opposite ends thereof in respective anchor elements and configured to form a loop extending from one of the anchor elements to the other one of the anchor elements, and an adjustment element engageable with respective legs of the loop for adjusting the effective length of the suture extending between the anchor elements.

193. A suture as claimed in claim 192 in which the adjustment element comprises a retainer configured for accommodating movement of at least one of the legs of the loop therethrough in a direction for urging the adjustment element along that leg of the loop towards the corresponding anchor element and for retaining the adjustment element engaged with that leg of the loop at a selected distance from the corresponding anchor element, and preferably, the retainer comprises a pair of spaced apart abutment members defining a suture accommodating gap therebetween for accommodating the at least one leg of the loop therethrough, and advantageously, the abutment members are configured to engage the at least one leg of the loop in the suture accommodating gap with an engagement force, and preferably, at least one of the abutment members is configured to engage the at least one leg of the loop in the suture accommodating gap with a friction grip, and advantageously, at least one of the abutment members is resiliently biased towards the other one thereof, and preferably, a gripping element is provided on at least one of the abutment members for gripping the at least one leg of the loop in the suture accommodating gap, the gripping element being configured to prevent movement of the adjustment element along the at least one leg of the loop in the direction away from the corresponding anchor element, and the gripping element being configured to permit movement of the adjustment element along the at least one leg of the loop in the direction towards the corresponding anchor element, and advantageously, the adjustment element comprises a first cross-member extending between and joining the abutment members, and preferably, first cross-member has a suture accommodating opening therethrough for accommodating the at least one leg of the loop into or out of the suture accommodating gap, and advantageously, the suture accommodating opening of each cross-member is configured to accommodate the two legs of the loop of the suture therethrough, and preferably, at least one of the abutment members defines one of an abutment face and an abutment edge for engaging the at least one leg of the loop, and advantageously, the suture accommodating gap defined between the pair of the abutment members is configured for accommodating the two legs of the loop of the suture therethrough, and preferably, the retainer is configured for accommodating the two legs of the loop of the suture therethrough, and alternatively,
the suture is severed intermediate the ends thereof, one leg of the suture adjacent the severed portion being non-adjustably secured to the adjustment element, and the other leg of the suture extending through the retainer of the adjustment element, and preferably, each end of the suture terminates in the corresponding transverse member intermediate the ends of the transverse member, and preferably, midway between the ends of the transverse member.

194. An adjustment element for a suture as claimed in claim 192.

195. A cannula having a bore extending longitudinally therethrough, and terminating adjacent a distal end thereof in a piercing point, and a suture accommodating chamber for accommodating a suture as claimed in claim 192 therein formed in the cannula bore.

196. A cannula as claimed in claim 195 in which the suture accommodating chamber is located adjacent the distal end of the cannula, and preferably, the suture is located in the suture accommodating chamber with the anchor elements thereof arranged sequentially longitudinally in the suture accommodating chamber, and advantageously, the length of the suture accommodating chamber is substantially equal to the sum of the lengths of the anchor elements of the suture, and preferably, a suture accommodating slot extends into the cannula from the distal end thereof, and advantageously, the proximal end of the cannula is configured for receiving an urging element into the cannula bore for urging the anchor elements of the suture from the suture accommodating chamber through the distal end of the cannula.

197. A cannula as claimed in claim 195 in which the proximal end of the cannula is configured for coupling to a first operating element of an actuator, and preferably, the proximal end of the cannula is configured for releasably coupling to the first operating element of the actuator, and advantageously, the proximal end of the cannula is configured for coupling to a connecting element for connecting the cannula to the first operating element of the actuator, and preferably, the proximal end of the cannula is configured for releasably coupling to a connecting element for connecting the cannula to the first operating element of the actuator, and advantageously, the proximal end of the cannula is configured for coupling to an elongated tubular connecting member for connecting the cannula to the first operating element of the actuator.

198. An actuator of a suturing device, the actuator comprising a first operating element configured for releasably coupling to the cannula as claimed in claim 195, a second operating element having an urging element, or being coupleable with an urging element for extending into the cannula bore from the proximal end thereof when the cannula is coupled to the first operating element, the second operating element being operable for urging the urging element in a distal direction into the suture accommodating chamber of the cannula for urging the anchor elements of the suture sequentially therefrom through the distal end of the cannula.

199. An actuator as claimed in claim 198 in which the second operating element is configured for urging the urging element distally in the suture accommodating chamber through a first predefined distance for urging a distal most one of the anchor elements of the suture from the suture accommodating chamber through the distal end of the cannula, and preferably, the second operating element is configured for urging the urging element distally through a second predefined distance in the suture accommodating chamber for urging a proximal most one of the anchor elements of the suture from the suture accommodating chamber through the distal end of the cannula, and advantageously, the second operating element is operable from a first state thereof to a second state thereof for urging the urging element through the first predefined distance, and preferably, the second operating element is moveable from the first state thereof to the second state thereof, and advantageously, a first stop element is provided for terminating movement of the second operating element from the first state thereof to the second state thereof when the second operating element has reached the second state thereof, and preferably, the second operating element is operable from the second state thereof to a third state thereof for urging the urging element through the second predefined distance, and advantageously, the second operating element is moveable from the second state thereof to the third state thereof, and preferably,
a second stop element is provided for terminating movement of the second operating element from the second state thereof to the third state thereof when the second operating element has reached the third state thereof, and advantageously, the second operating element is urgeable from the first state thereof to the second state thereof through a third predefined distance for urging the urging element distally through the first predefined distance, and preferably, the second operating element is urgeable from the second state thereof to the third state thereof through a fourth predefined distance for urging the urging element distally through the second predefined distance, and advantageously, the second operating element is moveable relative to the first operating element between the first and second states of the second operating element, and preferably, the second operating element is moveable relative to the first operating element between the second and third states of the second operating element, and advantageously, the second operating element is moveable in a first direction from the first state thereof to the second state thereof, and preferably, the second operating element is moveable from the second state thereof to the third state thereof in the first direction, and advantageously, the actuator further comprises a carrier for carrying the first and second operating elements, and preferably, the carrier comprises a first carrier element and a second carrier element extending distally from the first carrier element, and advantageously, the first carrier element is configured for carrying the first and second operating elements, and preferably, the second carrier element is configured for accommodating the cannula therein, and advantageously, the second carrier element is configured for slideably accommodating the cannula therein, and preferably, the first operating element is moveable from a first state thereof to a second state thereof for urging the cannula distally from a withdrawn state located within the second carrier element to an extended state with the cannula extending distally outwardly from a distal end of the second carrier element, and advantageously, the first operating element is slideable relative to the first carrier element between the first and second states thereof, and preferably, an engagement element is provided for engaging a portion of the suture and for urging the portion of the suture through an adjustment element of the suture, and advantageously, the engagement element is connectable to one of the first and second operating elements, and preferably, the engagement element is releasably connectable to the one of the first and second operating elements.

200. An actuator as claimed in claim 198 in which the engagement element is urgeable by the one of the first and second operating elements distally outwardly through the distal end of the second carrier element for engaging the portion of the suture, and preferably, the engagement element is urgeable in a proximal direction relative to the second carrier element by the one of the first and second operating elements for urging the portion of the suture through the adjustment element with the adjustment element abutting an abutment element of the actuator, and advantageously, the abutment element is defined by the distal end of the second carrier element, and preferably, the engagement element is connectable to the second operating element through the urging element, and advantageously,
the engagement element is located adjacent the distal end of the urging element, and preferably, the urging element is urgeable through a fifth predefined distance to the extended state after the second predefined distance for urging the engagement element distally through the distal end of the cannula and outwardly therefrom, and advantageously, the urging element is urgeable sequentially through the first, second and fifth predefined distances.

201. An actuator as claimed in claim 198 in which the second operating element is operable from the third state thereof to a fourth state thereof for urging the urging element distally through the cannula through a fifth predefined distance for urging the engagement element through the distal end of the cannula and outwardly therefrom, and preferably, the second operating element is moveable from the third state thereof to the fourth state thereof through a sixth predefined distance for urging the urging element distally through the fifth predefined distance, and advantageously, a third stop element is provided for terminating movement of the second operating element from the third state thereof to the fourth state thereof when the second operating element has reached the fourth state thereof, and preferably, the second operating element is moveable from the third state thereof to the fourth state thereof in the first direction, and advantageously, the second operating element is moveable in a second direction from the second state thereof to the first state thereof, and preferably, the second operating element is moveable in the second direction from the third state thereof to the second state thereof, and advantageously, the second operating element is urgeable from the fourth state thereof to the third state thereof in the second direction, and preferably, the second direction is opposite to the first direction.

202. An actuator as claimed in claim 198 in which a guide means is provided for guiding movement of the second operating element relative to the first operating element from the first state thereof to the second state thereof, and preferably, the guide means is adapted to guide movement of the second operating element from the second state thereof to the third state thereof, and advantageously,
the guide means is adapted to guide the movement of the second operating element from the third state thereof to the fourth state thereof, and preferably, the guide means is adapted to guide the second operating element to move relative to the first operating element in a third direction different to the first direction between travel of the second operating element through the third predefined distance and the fourth predefined distance, and advantageously, the guide means is adapted to guide the second operating element relative to the first operating element in the third direction or a fourth direction different to the first direction between travel of the second operating element through the fourth predefined distance and the sixth predefined distance, and preferably, the third direction is in a direction transversely of the first direction, and advantageously, the third direction of movement of the second operating element relative to the first operating element is in a circumferential direction relative to the first operating element, and preferably, the fourth direction is in a direction transversely of the first direction, and advantageously,
the fourth direction of movement of the second operating element relative to the first operating element is in a circumferential direction relative to the first operating element, and preferably, the fourth direction is in a direction of one of the same direction as the third direction of travel and opposite to the third direction of travel, and advantageously, the guide means comprises a camming means, and preferably, the camming means comprises a camming groove located on one of the first and second operating elements, and a cam follower located on the other one of the first and second operating elements, and advantageously, the first operating element is keyed relative to the first carrier element for preventing rotation of the first operating element on the first carrier element, and preferably, the first operating element is coaxial with the first carrier element, and advantageously, the second operating element is coaxial with the first operating element, and preferably, the urging element comprises an elongated push rod coupleable to the second operating element, and advantageously, an elongated tubular connecting member is coupleable to the first operating element, and preferably, the tubular connecting member terminates in a coupling element for coupling the cannula to the tubular connecting member, and advantageously, the urging element extends through the tubular connecting member, and preferably, the engagement element is coupleable to the tubular connecting member, and advantageously, the engagement element is releasably coupleable to the tubular connecting member, and preferably, the engagement element is coupleable to the urging element, and advantageously, the engagement element is releasably coupleable to the urging element.

203. A suturing device comprising an actuator as claimed in claim 198.

204. A method for suturing first and second parts of a lumen, vessel or organ in a cavity of a human or animal body using the suturing device as claimed in claim 184, the method comprising urging the cannula into the cavity or into the lumen, vessel or organ with a suture located in the suture accommodating chamber of the cannula, the suture terminating at respective opposite ends thereof in respective anchor elements and configured to form a loop extending from one of the anchor elements to the other one of the anchor elements, and an adjustment element engageable with respective legs of the loop for adjusting the effective length of the suture extending between the anchor elements, the method further comprising urging the distal piercing tip of the cannula through the tissue of the first part in the lumen, vessel or organ, urging the urging element distally into the suture accommodating chamber for urging a distal most one of the anchor elements of the suture through the distal end of the cannula, withdrawing the cannula from the first part in the lumen, vessel or organ leaving the suture extending through the first part from the anchor element from the distal most one of the anchor elements, urging the distal piercing point of the cannula through the second part in the lumen, vessel or organ, urging the urging element distally in the suture accommodating chamber for urging the proximal most one of the anchor elements through the distal end of the cannula, withdrawing the cannula from the second part of the lumen, vessel or organ, leaving the suture extending through the second part from the second anchor element, engaging the engagement element with the suture and urging the suture through the adjustment element of the suture for reducing the effective length of the suture between the anchor elements to pull the first and second parts to be sutured together.

205. A method as claimed in claim 204 in which the engagement element is disengaged from the suture after the first and second parts of the lumen, vessel or organ have been urged together, and the cannula is withdrawn from the cavity or the lumen, vessel or organ, and preferably, the adjustment element of the suture is urged against the abutment element as the suture is being urged through the adjustment element by the engagement element, and advantageously, the cannula is initially located within the second carrier element in the withdrawn state, and the second carrier element is urged into the cavity or the lumen, vessel or organ before the cannula is urged from the withdrawn state through the distal end of the second carrier element, and preferably, the engagement element is urged distally outwardly from the cannula prior to engaging the portion of the suture to be urged through the adjustment element, and advantageously, the engagement element is urged into the second carrier element for drawing the portion of the suture into the second carrier element with the adjustment element abutting the abutment element defined by the distal end of the second carrier element for urging the portion of the suture through the adjustment element, and preferably, the method is configured for suturing first and second parts of a colon wall of a colon on respective opposite sides of a polyp together, for everting the polyp so that the polyp extends from an external side of the colon wall.
